(19) United States
(12) Patent Application Publication
(10) Pub. No.: US 2022/0370488 A1
(43) Pub. Date: Nov. 24, 2022

(54) MUCUS PENETRATING PARTICLE COMPOSITIONS AND METHODS OF USE THEREOF ENHANCING IMMUNE RESPONSE

(71) Applicant: The Johns Hopkins University, Baltimore, MD (US)

(72) Inventors: Jung Soo Suk, Baltimore, MD (US); Yoo Chun Kim, Baltimore, MD (US); Justin Hanes, Baltimore, MD (US)

(21) Appl. No.: 17/770,437

(22) PCT Filed: Oct. 21, 2020
(86) PCT No.: PCT/US2020/056590

§ 371 Date: Apr. 20, 2022

Related U.S. Application Data
(60) Provisional application No. 62/924,460, filed on Oct. 22, 2019.

(51) Int. Cl. A61K 31/711; A61K 47/69; A61K 9/00; A61K 9/51

(52) U.S. Cl. CPC A61K 31/711; A61K 47/6935; A61K 9/0034; A61K 9/0053; A61K 9/5107

(57) ABSTRACT

Mucus penetrating nanoparticles for inducing, increasing, or enhancing an immune response typically include core of a blend of a biodegradable hydrophobic polymer and a hydrophilic polymer, wherein ≥50% of the biodegradable polymer is conjugated to the hydrophilic polymer, and the hydrophilic polymers forms a coating on the particle. The particles encapsulate a cargo, typically an antigen, adjuvant or other immunomodulator, or a nucleic acid encoding the antigen, or combination thereof. Pharmaceutical compositions including an effective amount of particles to induce an immune response in a subject in need thereof are also provided. Methods of inducing an immune response are also provided, and typically include administering to a subject, preferably via the respiratory tract, the pharmaceutical composition. In some embodiments, the subject has cancer or an infection of the lung.

### CROSS-REFERENCE TO RELATED APPLICATION

This application claims the benefit of and priority to U.S. Ser. No. 62/924,460 filed Oct. 22, 2019 and which is incorporated by referenced herein in its entirety.

### STATEMENT REGARDING FEDERALLY SPONSORED RESEARCH OR DEVELOPMENT

This invention was made with government support under grants HL127413 and EY001765 awarded by the National Institute of Health. The government has certain rights in the invention.

### FIELD OF THE INVENTION

The invention is generally directed to the field of molecular delivery systems, and more specifically, to mucus penetrating particles for the delivery of nucleic acids, antigens, or small molecules to a subject to prevent or treat diseases and/or conditions.

### BACKGROUND OF THE INVENTION

Human lung airways from the trachea to bronchioles are rich in dendritic cells (DC) that persistently sample inhaled foreign matters deposited on the airway lumen to initiate antigen specific immune responses (Thornton, et al., J. Exp. Med., 209:1183-1199 (2012)). Pulmonary vaccination provides a straightforward means to combat against inhaled pathogens, including influenza and Mycobacterium tuberculosis, or to eradicate lung cancers if immunologically active. Pulmonary vaccination elicits stronger immune responses in the lung and is more efficient in clearing respiratory pathogens compared to other systemic immunization strategies (Belyakov, et al., Nat. Med., 7:1320-1326 (2001)).

Inhaled DNA vaccination, by promoting both humoral and cellular immunity, can potentially manage numerous lung diseases both in prophylactic and therapeutic manners (Kutzler, et al., Nat. Rev. Genet., 9:776-788 (2008); Rice, et al., Nat. Rev. Cancer, 8:108-120 (2008)). Of note, DNA vaccine holds additional advantageous features in comparison to traditional subunit vaccines, including ease and speed of scale-up, superior stability and potential of global distribution without a need of expensive and cumbersome ‘cold-chain’ (DeMuth, et al., Nat. Mater., 12:367-376 (2013); Kim, et al., Adv. Drug Deliv. Rev., 64:1547-1568 (2012)).

Nevertheless, clinical trials of DNA vaccination targeting lung diseases to date have primarily explored conventional systemic approaches (e.g., intramuscular electroporation) (DeMuth, et al., Nat. Mater., 12:367-376 (2013); Lee, et al., Front. Immunol., 9:1568 (2018)). The field of DNA vaccination has been primarily focusing on strategies to enhance DC uptake of DNA vaccines, such as molecular targeting and electroporation (Tacken, et al., Nat. Rev. Immunol., 7:790-802 (2007); van Broekhoven, et al., Cancer Res., 64:4357-4365 (2004)).

However, a largely overlooked challenge to inhaled vaccination is the mucus gel layer lining the lung airways, which may hamper the access of inhaled DNA vaccines to pulmonary DC residing in the airway mucosa. The airway mucus is a protective barrier that effectively traps inhaled foreign matters, including large DNA (e.g., plasmid-based DNA vaccines) (Sanders, et al., J. Control. Release, 87:117-129 (2003)), via adhesive and/or physical interactions, and rapidly clears them from the lung via the physiological mucociliary clearance (MCC) mechanism (Duncan, et al., Mol. Ther., 24:2043-2053 (2016); Kim, et al., J. Control. Release, 240:465-488 (2016)). While gene transfer agents (i.e., gene vectors) have been shown to improve diffusion of DNA in mucus to a certain degree (Shen, et al., Biophys. J., 91:639-644 (2006)), conventional virus-based and synthetic gene vectors, including those tested in clinical trials, cannot efficiently penetrate human airway mucus (Sanders, et al., J. Control. Release, 87:117-129 (2003); Hida, et al., PLoS One, 6:e19919 (2011); Suk, et al., J. Control. Release, 178:8-17 (2014)). To this end, these gene vectors, following inhaled administration, are unlikely to shuttle DNA vaccine payloads efficiently to pulmonary DC prior to the MCC to induce a robust immune response in the lung.

There remains a need for strategies to efficiently deliver DNA vaccine components to pulmonary DC to mediate strong and durable immunity in the lung and other mucosal surfaces.

Therefore, it is the object of the present invention to provide improved immunogenic compositions, and method of use thereof.

### SUMMARY OF THE INVENTION

Mucus penetrating nanoparticles for inducing, increasing, or enhancing an immune response are provided. The particles typically include a blend of a biodegradable polymer and a hydrophilic polymer, wherein ≥50% of the biodegradable polymer is conjugated to the hydrophilic polymer. The nanoparticle is coated with the hydrophilic polymer. The mass ratio of the biodegradable polymer to the conjugated polymer can be, for example, between 0.5 and 1, based on the mass of the biodegradable polymer. In some embodiments, the hydrodynamic diameter of the nanoparticle is 100 nm or less. The surface charge of the nanoparticle can be near neutral. In some embodiments, the biodegradable polymer is poly(β-amino ester) with a molecular weight between 4 kDa and 7 kDa. The hydrophilic polymer can be, selected from, for example, polyethylene glycol, polyethylene oxide, and copolymers thereof. In some embodiments, the hydrophilic polymer is polyethylene glycol with a molecular weight between 1 kDa and 10 kDa.

The particles encapsulate a cargo, most typically an immunological cargo such as an antigen, adjuvant, or other immunomodulator, or a nucleic acid encoding the same. In some preferred embodiments, the cargo is a nucleic acid encoding a polypeptide antigen. The nucleic acid can be DNA or RNA. In preferred embodiments, the nucleic acid is a DNA vector encoding a heterologous expression control sequence operably linked to a sequence encoding the polypeptide antigen. The vector can be, for example, a plasmid or a viral vector. In some embodiments, the mass ratio of the blended polymer to the nucleic acid is up to 100.

The antigen is typically a polypeptide immunogen capable of inducing an immune response in a subject in need thereof. The antigen can be a T cell antigen. In some embodiments, the nanoparticle alternatively or additionally encapsulates an adjuvant, preferably a molecular adjuvant.

Exemplary adjuvants, including molecular adjuvants, are also provided and include, for example, ligands for pattern recognition receptors (PPRs), adaptor proteins, inflammation singling proteins, transcription factors, cytokines, chemokines, immune costimulatory molecules, toll-like receptor agonists or inhibitors of immune suppressive pathways, and immune regulators, or nucleic acids encoding same. In some embodiments, the adjuvant is a ligand for a PPR, for example a ligand for a Toll-like family member. In some embodiments, the adjuvant acts through TLR3, TLR4, TLR5, TLR6, TLR7, TLR8, TLR9, or a combination thereof. The adjuvant can be an oligonucleotide, for example, an oligonucleotide including one or more unmethylated cytosine-guanine (CpG) dinucleotide motifs. The adjuvant can Poly(I:C) of a derivative thereof.

Alternatively or additionally, the nanoparticles can encapsulate one or more immunomodulatory agents. Such agents include, but are not limited to, synthetic receptor ligands, proteins, cytokines, interleukins, tumor necrosis factor, and combinations thereof.

Pharmaceutical compositions including an effective amount of nanoparticles and a pharmaceutically acceptable carrier are also provided.

Preferably, the pharmaceutical compositions are immunogenic. The nanoparticles can be in an amount effective to induce an immune response in a subject in need thereof. In some embodiments, upon administration to a subject in need thereof, the composition increases antigen uptake in pulmonary DC (CD11⁺CD170⁻); increases DC maturation; increases DC number or frequency, particularly pulmonary DCs (CD11c⁺CD170⁻) in the lung airway interstitium; increases DC migration to the lymph nodes; increases antigen-specific CTL response, particularly in the lung, mediastinal LN and/or spleen; increases activated CD8⁺ T-cells (IFN-g⁺CD8⁺) and/or increases frequencies of CD4⁺ T-cell activation, particularly in the lung, mediastinal LN and/or spleen; increases dissemination of antigen-specific CD8⁺ T cells to, and/or CTL responses in, tissues distal to the site of administration; increases antigen specific T-cell memory biased towards the effector memory phenotype both at the site of administration and/or systemically in the spleen, preferably wherein the bias is most prominent in the lung; or a combination thereof.

In some embodiments, the nanoparticles are formulated for administration to a mucosal layer. The experiments below illustrate that when delivered to the lungs, the nanoparticles can be taken up by pulmonary DC and subsequently traffic to lymph node. In some embodiments, adjuvant is loaded into the same nanoparticles as the antigen or nucleic acid encoding the antigen, into different nanoparticles from the antigen or nucleic acid encoding the antigen, or a combination thereof. In some embodiments, adjuvant is not loaded into nanoparticles.

Methods of inducing an immune response are also provided. Typically, the methods include administering to the respiratory tract of a subject an immunogenic composition of nanoparticles. In some embodiments, the composition is administered to a mucosal layer. In preferred embodiments, the composition increases adaptive immunity in the lung and other remote mucosal surfaces, for example, the gastrointestinal tract, vaginal tract, or a combination thereof. In some embodiments, the composition increases systemic immunity. In preferred embodiments, the composition increases systemic immunity to a level greater than the dose-matched immunogenic cargo administered via routes commonly applied for systemic immunization, such as intramuscular immunization. In some embodiments, the subject has cancer or an infection, and the immune response is against the cancer or infection. Thus, methods of treating and preventing cancers and infections are also provided.

### BRIEF DESCRIPTION OF THE DRAWINGS

FIGS. 1A and 1B are representative flow cytograms demonstrating pOVA-MPP (FIG. 1A) and pOVA-CP (FIG. 1B) in vivo uptake by pulmonary DC at 16-hour post-administration. FIG. 1C is a bar graph showing the percentages of pulmonary DC that took up pOVA-MPP and pOVA-CP at 16-hour post-administration (n=6). ***p<0.0005 by one-way analysis of variance (ANOVA).

FIGS. 2A-2C are representative flow cytograms demonstrating in vitro DC uptake of different pOVA-loaded nanoparticles, including untreated (FIG. 2A), pOVA-MPP (FIG. 2B) and pOVA-CP (FIG. 2C). FIG. 2D is a graph showing the percentage of DC that took up pOVA-MPP and pOVA-CP at 4-hour post-incubation (n=3). *p<0.05 and ***p<0.0005 by ANOVA.

FIGS. 3A and 3B are bar graphs showing changes in hydrodynamic diameters (FIG. 3A) and polydispersity index (PDI) values (FIG. 3B) of pOVA-MPP, p(I:C)/pOVA-MPP, and CpG/pOVA-MPP in PBS over 6 hours (n=3-9). FIG. 3C is a dot plot showing the median MSD of pOVA-MPP, p(I:C)/pOVA-MPP, and CpG/pOVA-MPP in freshly collected human airway mucus samples (n≥3) at a timescale of 1 s. The MSD values are directly proportional to particle diffusion rates. FIG. 3D is a bar graph showing the percentage of DC11c⁺ DC co-expressing MHC-II⁺ and CD86⁺ following a 6-hour incubation with various adjuvants or particle formulations (n≥5). *p<0.05, **p<0.005, and ***p<0.0005 by ANOVA.

FIG. 4A is an illustration of an experimental immunization schedule. FIGS. 4B-4D are bar graphs showing the percentage of CD8⁺ T-cells (CD3_{ε}⁺ CD8⁺) expressing OVA-specific MHC-I peptide (SIINFEKL (SEQ ID NO:1)) in lung (FIG. 4B), respective draining LN (FIG. 4C), and spleen (FIG. 4D) following pOVA-mediated DNA vaccination via different administration routes. ID: intradermal; IM-EP: intramuscular electroporation; IT: intratracheal. *p<0.05 and ***p<0.0005 by ANOVA.

FIGS. 5A-5E are bar graphs showing the percentages of CD8⁺ (CD3_{ε}⁺ CD8⁺) and/or CD4⁺ (CD3_{ε}⁺ CD4⁺) T-cells harvested from the lung (FIGS. 5A and 5B), respective draining LN (FIG. 5C), and spleen (FIGS. 5D and 5E) expressing INF-γ after ex vivo re-stimulation (n≥5). ID: intradermal; IM-EP: intramuscular electroporation; IT: intratracheal. ***p<0.0005 by ANOVA.

FIGS. 6A-6C are bar graphs showing the percentage of CD8⁺ T-cells (CD3_{ε}⁺CD8⁺) expressing OVA-specific MHC-I peptide (SIINFEKL (SEQ ID NO:1)) in mesenteric LN (FIG. 6A), Payer's patch (FIG. 6B), and vagina (FIG. 6C) after immunization (n≥5).

FIG. 7 is a bar graph showing the percentage of CD8⁺ T-cells (CD3_{ε}⁺ CD8⁺) expressing gut-homing integrin α₄β₇ in mediastinal LN was determined by flow cytometry 7 days after intratracheal boost administration of CpG/pOVA or CpG/pOVA-MPP. **p<0.005, and ***p<0.0005 by ANOVA.

FIGS. 8A-8C are bar graphs showing the percentage of CD8⁺ T-cells (CD3_{ε}⁺CD8⁺) expressing OVA-specific MHC-I peptide (SIINFEKL (SEQ ID NO:1)) in mesenteric LN (FIG. 8A), Payer's patch (FIG. 8B), and vagina (FIG. 8C) with adoptive T-cell transfer (n=3-5). IT: intratracheal. *p<0.05, **p<0.005, and ***p<0.0005 by Student's t-test or ANOVA. CD8⁺ T-cells from OT-I mice were adoptively transferred into C57BL/6 mice one day prior to immunization with CpG/pOVA-MPP and OVA-specific T-cell response was quantified 3 days after the immunization. ID: intradermal; IM-EP: intramuscular electroporation; IT: intratracheal. *p<0.05, **p<0.005, and ***p<0.0005 by Student's t-test or ANOVA.

FIGS. 9A-9C are bar graphs showing the percentages of CD8⁺ T-cells (CD3_{ε}⁺CD8⁺) expressing OVA-specific MHC-I peptide (SIINFEKL (SEQ ID NO:1)) in lung (FIG. 9A), mediastinal LN (FIG. 9B), and spleen (FIG. 9C) 70 days after the pulmonary immunization with CpG/pOVA-MPP. IT: intratracheal. ***p<0.0005 by Student's t-test or ANOVA.

FIGS. 10A-10C are bar graphs showing the percentage of OVA-specific central memory T-cells (T_{CM}) co-expressing CD44^{hi} and CD62L^{hi} and effector memory T-cells (T_{EM}) co-expressing CD44^{hi} and CD62L^{lo} in lung (FIG. 10A), mediastinal LN (FIG. 10B), and spleen (FIG. 10C) 70 days after the pulmonary immunization with CpG/pOVA-MPP (n≥5). IT: intratracheal. *p<0.05 and **p<0.005 by Student's t-test or ANOVA.

FIG. 11A is an illustration of an experimental immunization schedule. C57BL/6 mice (an orthotopic mouse model of OVA-expressing lung cancer) were immunized as described in FIG. 4A and OVA-LLC cells were intratracheally inoculated into the lung 7 days after the boost. Mice received CpG/pOVA or CpG/pOVA-MPP via different administration routes. FIG. 11B is a Kaplan-Meier survival curve illustrating the results (n≥6). ID: intradermal; IM-EP: intramuscular electroporation; IT: intratracheal.

FIG. 12A is an illustration of an experimental immunization schedule to study the survival of tumor-bearing mice following sham administration. FIG. 12B is Kaplan-Meier survival curve illustrating the results (n=5).

### DETAILED DESCRIPTION OF THE INVENTION

### I. Definitions

As used herein, the term “antigen” is a molecule capable of being recognized or bound by an antibody or T-cell receptor. An “immunogen” is an antigen that is additionally capable of provoking an immune response against itself (e.g., upon administration to a mammal, optionally in conjunction with an adjuvant). This immune response can involve either antibody production, or the activation of specific immunologically-competent cells, or both. Any macromolecule, including virtually all proteins or peptides, can serve as an antigen or immunogen. Furthermore, antigens/immunogens can be derived from recombinant or genomic DNA. Any DNA that includes a nucleotide sequences or a partial nucleotide sequence encoding a protein or peptide that elicits an immune response therefore encodes an “immunogen” as that term is used herein. An antigen/immunogen need not be encoded solely by a full length nucleotide sequence of a gene. An antigen/immunogen need not be encoded by a “gene” at all. An antigen/immunogen can be generated, synthesized, or can be derived from a biological sample. Such a biological sample can include, but is not limited to a tissue sample, a tumor sample, a cell or a biological fluid.

As used herein, an “adjuvant” is a substance that increases the ability of an antigen to stimulate the immune system.

As used herein, the term “biodegradable” refers to a material that degrades or breaks down into its component subunits, or digestion, e.g., by a biochemical process, of the polymer into smaller, non-polymeric subunits.

As used herein, the term “immune cell” refers to a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, the term “T cell” refers to a CD4+ T cell or a CD8+ T cell. The term T cell includes TH1 cells, TH2 cells and TH17 cells.

As used herein, the term “T cell cytoxicity” includes any immune response that is mediated by CD8⁺ T cell activation. Exemplary immune responses include cytokine production, CD8⁺ T cell proliferation, granzyme or perforin production, and clearance of an infectious agent.

As used herein, the term “systemic immunity” refers to the immunity in spleen.

As used herein, the terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

As used herein, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that can have various lengths, either deoxyribonucleotides (DNA) or ribonucleotides (RNA), or analogs or modified nucleotides thereof, including, but not limited to locked nucleic acids (LNA) and peptide nucleic acids (PNA). An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides.

As used herein, the term “gene” refers to a nucleic acid (e.g., DNA or RNA) sequence that including coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides determines the order of amino acids along the polypeptide (protein) chain.

As used herein, “heterologous” means derived from a different species.

As used herein, “homologous” means derived from the same species. For example, a homologous trait is any characteristic of organisms that is derived from a common ancestor. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

As used herein, “autologous” means derived from self.

As used herein, “endogenous” means a substance that originates from within an organism, tissue, or cell.

As used herein, “exogenous” means a substances that originates from outside an organism, tissue, or cell.

As used herein a “recombinant protein” is a protein derived from recombinant DNA.

As used herein “recombinant DNA” a refers to DNA molecules that is extracted from different sources and chemically joined together; for example DNA including a gene from one source may be recombined with DNA from another source. Recombinant DNA can be all heterologous DNA or a combination of homologous and heterologous DNA. The recombinant DNA can be integrated into and expressed from a cell's chromosome, or can be expressed for an extra-chromosomal array such as a plasmid.

As used herein, “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including for example: attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition. For example, at least two actives can be encapsulated. In another example, at least three, at least four, at least five or more actives can be encapsulated.

More specifically, the physical form in which any therapeutic agent or other material is encapsulated in polymers may vary with the particular embodiment. For example, a therapeutic agent or other material may be first encapsulated in a sphere and then combined with the polymer in such a way that at least a portion of the sphere structure is maintained. Alternatively, a therapeutic agent or other material may be sufficiently immiscible in the polymer that it is dispersed as small droplets, rather than being dissolved, in the polymer.

As used herein, the term “corresponding particle”, “conventional particle” or “reference particles” refers to a particle that is substantially identical to another particle to which it is compared, but typically lacking a surface modification to promote effective compaction, colloidal stability and transport differences through the pores of airway mucus and of the extracellular matrix (ECM) of the brain. In certain embodiments, a corresponding particle is a particle that does not have a dense coating of polyethylene glycol. In certain embodiments, a comparable particle is a particle that is not formed of a blended mixture containing free polymer and polymer conjugated to polyethylene glycol.

As used herein, the term “densely coated particle” refers to a particle that is modified to specifically enhance the density of coating agent at the surface of the particle, for example, relative to a reference particle. In some embodiments, a densely coated particle is formed from a ratio of polyethylene glycol to polymer that is sufficient to alter the physicochemical properties of the particle relative to a less densely coated, or non-coated particle. In some embodiments, the density of coating agent is sufficient to completely mask the charge of the particle, resulting in a near neutral charge and near neutral zeta potential value and colloidal stability in physiological solutions. In a particular embodiment, a densely coated particle is achieved using branched polyethylene glycol or branched polymer, wherein the branching enhances the ratio of polyethylene glycol to polymer as compared to a reference particle that does not contain a branched polymer or branched polyethylene glycol.

As used herein, the term “effective amount” or “therapeutically effective amount” means a dosage sufficient to provide treatment for a disorder, disease, or condition being treated, to induce or enhance an immune response, or to otherwise provide a desired pharmacologic and/or physiologic effect. The precise dosage will vary according to a variety of factors such as subject-dependent variables (e.g., age, immune system health, etc.), the disease, the disease stage, and the treatment being effected.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%.

### II. Compositions

A. Mucus-Penetrating NanoParticles (MPPs)

Delivery particles with a dense surface coating of hydrophilic polymer, capable of rapid diffusion and widespread distribution through mesh-like biological barriers, such as the airway mucus and mucosal layers in gastrointestinal (GI) and vaginal tracts, are disclosed.

The mucus-penetrating nanoparticles (MPPs) formed from polymers such as poly (β-amino ester) polymers (PBAE) conjugated to hydrophilic polymers such as polyethylene glycol (PEG) provide a non-toxic, biodegradable polymer library for the compaction of nucleic acids, offering highly effective gene delivery in vitro and in vivo. The MPPs can rapidly penetrate airway mucus, leading to efficient access to and uptake by pulmonary dendritic cells (DC).

The MPP includes a blend of a biodegradable polymer and a coating agent such as a hydrophilic polymer, where at least 10%, 25%, or 50% of the biodegradable polymer is conjugated to the coating agent. The MPP has a polymer core formed from the biodegradable polymer, which is coated with the coating agent. The MPP also contains a cargo, typically polypeptide antigen expressing nucleic acids, or the polypeptide antigen itself, and optionally an adjuvant and/or other immunomodulatory agents encapsulated therein or associated with the surface of the MPPs. In some embodiments, the adjuvant is a nucleic acid-based adjuvant. In preferred embodiments, the biodegradable polymer is a cationic polymer. Typically, at least 50% of the biodegradable polymer is conjugated to the coating agent.

The polymers can be synthesized using semi-automated high-throughput combinatorial chemistry offering a large variety of polymers ((Akinc, et al., Bioconjug Chem, 2003. 14(5): 979-88)) for the formulation of gene vectors with different chemical properties, while providing high density surface PEG coatings.

PEGylation of cationic polymers may have negative influences on nucleic acids complexation due to reduction of available positive charges resulting from the PEG conjugation to the amine groups of cationic polymers and additional steric hindrance imposed by grafted PEG chains. To overcome this limitation and achieve dense PEG surface coating, a non-PEGylated polymer core was used for compact nucleic acids complexation.

In some embodiments, the blended polymer contains a mass ratio of free biodegradable polymer (or “free polymer”) to biodegradable polymer conjugated to hydrophilic polymer (or “conjugated polymer”) of between 0.1 and 1, between 0.25 and 1, or between 0.5 and 1, for example, about 0.67, based on the mass of the biodegradable polymer. An exemplary biodegradable polymer is poly (β-amino ester) polymer (PBAE). In some embodiments, the mass ratio of the PBAE polymer in the blended polymer to cargo is up to 100, for example, about 60. In preferred embodiments, the cargo is antigen expressing nucleic acids and adjuvants. In some embodiments, the mass ratio of antigen expressing nucleic acid to adjuvant is up to 10, for example, about 4. In preferred embodiments, the adjuvant is a nuclei acid-based adjuvant and the mass ratio of antigen expressing nucleic acid to nucleic acid-based adjuvant is up to 10, for example, about 4.

Exemplary MPPs for delivery of cargo across biological barriers include cargo such as antigen expressing nucleic acids, adjuvants, immunomodulatory agents, or a combination thereof, poly (β-amino ester) polymer, and hydrophilic polymer. At least 10%, 25%, or 50% of the poly (β-amino ester) in the particles is conjugated to the hydrophilic polymer and the cargo encapsulated within the particles or are associated with the surface of the nanoparticles. The particles are coated with the hydrophilic polymer at a density that imparts a near neutral surface charge, and have a diameter of less than 100 nm. Typically, the poly (β-amino ester) has a molecular weight greater than 2,000 Daltons, for example, 7,000 Daltons. In some embodiments, the hydrophilic polymer is polyethylene glycol (PEG) that has a molecular weight between 1,000 Daltons and 10,000 Daltons, for example, 5,000 Daltons.

Effective nucleic acids compaction can be achieved using a mixture of biodegradable polymers conjugated with a hydrophilic, neutrally charged polymer and non-conjugated biodegradable polymers (free polymers). Formulation parameters, such as biodegradable polymer/nucleic acids weight ratio in the blended polymer, free polymer/conjugated polymer ratio, pH of nucleic acid and blended polymer solutions, type of buffering solution and method of mixing can be optimized to increase stability and transfection efficiency. These MPPs retain their physicochemical characteristics, including hydrodynamic diameter, polydispersity index and surface charge, over at least 6 hours, 24 hours, 3 days, or a week in aqueous solution and post-lyophilization. They are also highly stable in physiological solutions, such as bronchoalveolar laveage fluid (BALF) and artificial cerebrospinal fluid (aCSF). For example, the hydrodynamic diameter of the MPPs, upon incubation in a bronchoalveolar lavage fluid (i.e., physiological lung fluid), changes less than 25% within 4 hours.

The dense coating of a hydrophilic polymer, indicated by their near neutral surface charge, in combination with their relatively small diameter (<100 nm) allows them to rapidly penetrate through mesh-like biological barriers such as the airway mucus and mucosal layers in GI and virginal tracts. These attributes offer a window of opportunity for in vivo nucleic acids delivery to different organs, especially via inhaled administration route. For example, the MPPs address a leading limitation to pulmonary DNA vaccination via inhaled administration route: limited access to and uptake by pulmonary DC due to the protective airway mucosal barrier. These inhaled MPPs also provide significantly greater systemic immune responses compared to gold-standard approaches applied in clinic for systemic vaccination. For example, inhaled MPPs containing antigen expressing plasmids and adjuvants can provide greater systemic immunity than dose-matched antigen expressing plasmids and adjuvants co-administered via routes commonly applied for systemic immunization, such as intramuscular immunization.

1. Coating Agents

The particles typically include a coating agent. Typically, the surface-altering coating agents impart a near-neutral negative charge and promote penetration and diffusion of the particles through biological barriers. The coating agents can minimize interactions with the highly adhesive and electrostatically charged components of mesh like biological barriers, such as the airway mucus, mucosal layers of GI and vaginal tracts, and tumor tissue.

Examples of coating polymers include polyalkylene oxides and copolymers thereof, including poly(ethylene glycols) (“PEG”) and poloxomers (polyethylene oxide block copolymers).

A preferred coating agent is PEG. PEG may be employed to improve compaction, enhance stability and reduce adhesion in mucus in the body in certain configurations, e.g., the length of PEG chains extending from the surface is controlled (such that long, unbranched chains that interpenetrate into the mucus are reduced or eliminated).

Representative PEG molecular weights in daltons (Da) include 300 Da, 600 Da, 1 kDa, 2 kDa, 3 kDa, 4 kDa, 5 kDa, 6 kDa, 8 kDa, 10 kDa, 15 kDa, 20 kDa, 30 kDa, 50 kDa, 100 kDa, 200 kDa, and 500 kDa. In some embodiments, the PEG has a molecular weight between 1 kDa and 10 kDa, for example, 2 kDa, 3.4 kDa, or 5 kDa. In preferred embodiments, the PEG has a molecular weight of about 5 kDa. PEG of any given molecular weight may vary in other characteristics such as length, density, and branching. In preferred embodiments, a coating agent is methoxy-PEG-amine, with a MW of 5 kDa. In some embodiments, a coating agent is methoxy-PEG-N-hydroxysuccinimide with a MW of 5 kDa (mPEG-NHS 5 kDa).

The preferred range is 2 kD (Huang, X. L. et al. Proc. Natl Acad Sci USA 114, E6595-E6602, doi:10.1073/pnas.1705407114 (2017)), 3.4 kD (Suk, J. S. et al. Biomaterials 30, 2591-2597, doi:10.1016/j.biomaterials.2008.12.076 (2009)), and 5 kDa (Mastorakos, P. et al. Proc Natl Acad Sci USA 112, 8720-8725, doi:10.1073/pnas.1502281112 (2015)). Dense surface coatings with 2, 3.4, and 5 kDa PEG render different types of nanoparticles capable of efficiently penetrating airway mucus.

In the preferred embodiment, PEG is covalently bound to the PBAE polymer via succinimidyl succinate in PEG molecule reacting with amine groups at the terminal ends of PBAE polymer.

In preferred embodiments, the nanoparticles are coated with PEG or another hydrophilic coating agent at a density that imparts a near neutral surface charge. The density of the coating can be varied based on a variety of factors including the material and the composition of the particle.

In preferred embodiments, the molar ratio of PEG or other coating agent to cationic polymer such as PBAE for formulation of the PEG-PBAE co-polymer is equal to or greater than 2. In the nanoparticle formulated using a blended strategy, the mass ratio of PEG to PBAE is equal to or greater than 0.5. The ratio by mass of PEG or other coating agent to cationic polymer can be 0.5, 1, 2,3, 4, 5, 6, 7, 8, 9, or 10. In some embodiments, the density of the PEG or other coating agent is at least 0.01, 0.02, 0.05, 0.1, 0.2, 0.5, 1, 2, 5, 10, 20, 50, or 100 units per nm².

For example, to synthesize PEGylated PBAE, approximately 4 kDa PBAE polymers were synthesized and two 5 kDa PEG polymers conjugated to both ends of the PBAE polymer. In parallel, non-PEGylated PBEA polymer possessing MW of approximately 6 kDa was synthesized. A blend of PEGylated and non-PEGylated PBAE polymer was then prepared at a mass ratio of 6:4 based on the relative mass of PBAE, to produce a PEG:PBAE mass ratio in the blend of 3:2. This blend is then used to package nucleic acids into nanoparticles. The amount and ratio of polymers used for the particles are identical to those reported in Mastorakos 2015 and US20170072064A1, these particles have small nucleic acid-based adjuvantsco-packaged with (antigen-encoding) plasmid DNA, in contrast to the previous formulations including only plasmid DNA.

2. Biodegradable Core Polymers

In preferred embodiments, the biocompatible polymer(s) is a cationic polymer. Typically, the biocompatible polymer(s) is biodegradable.

### i. Poly(β-Amino Ester)

In preferred embodiments, the core polymer is poly (β-amino ester) (PBAE). PBAEs, when added to pH 5 buffer, are positively charged and can spontaneously form positively-charged nanoparticles (generally less than 200 nm) when added to negatively charged nucleic acid. They are taken up via endocytosis, and enable endosomal escape by buffering the endosome. PBAE can be readily degraded by hydrolysis of the ester bonds in the polymer backbone, enabling reduced cytotoxicity when compared to non-degradable controls. Modification of the polymer ends of PBAE can further improve transfection efficiency. PBAEs can provide a non-toxic, biodegradable polymer library for the compaction of DNA, offering highly effective gene delivery in vitro even in cells that are hard to transfect.

PBAEs can be synthesized using semi-automated high-throughput combinatorial chemistry offering a large variety of polymers for the formulation of gene vectors with different properties. PBAE core polymers of different molecular weights can be used to formulate nanoparticle gene carriers. Representative PBAE polymers include PBAE with a molecular weight of 1 kilo-Dalton (kDa), 2 kDa, 3 kDa, 4 kDa, 5 kDa, 6 kDa, 8 kDa, 9 kDa 10 kDa, 11 kDa, 12 kDa, 13 kDa, 14 kDa, 15 kDa, and more than 15 kDa.

Methods for synthesizing PBAE of different molecular weights are known in the art. For example, PBAE can be synthesized by reacting 1,4-butanediol diacrylate and 4-amino-1-butanol at the molar ratios of 1.2:1 (PBAE_{low}, MW˜4 kDa), 1.1:1 (PBAE_{mid}, MW˜7 kDa) and 1.05:1 (PBAE_{high}, MW˜11 kDa) while stirring at 90° C. for 24 hours. Polymers can be precipitated and washed in cold ether and dried under vacuum. The molecular weights of the PBAE base polymers can be made by any methods known in the art, including gel permeation chromatography and nuclear magnetic resonance spectroscopy.

### ii. Other Polymers

Other polymers, including biodegradable and bioreducible polymers, may be used to produce the MPPs. A representative list of polymers that can be used includes cyclodextrin-containing polymers, in particular cationic cyclodextrin-containing polymers, such as those described in U.S. Pat. No. 6,509,323, polyethylenimine (PEI), poly(L-lysine) (PLL), polymethacrylate, chitosan, poly(glycoamidoamine), schizophyllan, DEAE-dextran, dextran-spermine, poly(amido-amine) (PAA), poly(4-hydroxy-L-proline ester), poly[R-(4-aminobutyl)-L-glycolic (PAGA), poly(amino-ester), poly(phosphazenes) (NW, poly(phosphoesters) (PPE), poly(phosphoramidates) (PPA), TAT-based peptides, Antennapedia homeodomain peptide, MPG peptide, poly(propyleneimine), carbosilane, amine-terminated polyaminophosphine. In some embodiments, the polymer is a cationic polymer with multiple free amines. Suitable polymers include polyethylenimine (PEI) and poly-L-lysine (PLL).

Copolymers of two or more polymers described above, including block and/or random copolymers, may also be employed to make the polymeric particles.

### iii. Branched Polymers

In polymer chemistry, branching occurs by the replacement of a substituent, e.g., a hydrogen atom, on a monomer subunit, by another covalently bonded chain of that polymer; or, in the case of a graft copolymer, by a chain of another type. Branching may result from the formation of carbon-carbon or various other types of covalent bonds. Branching by ester and amide bonds is typically by a condensation reaction, producing one molecule of water (or HCl) for each bond formed.

The branching index measures the effect of long-chain branches on the size of a macromolecule in solution. It is defined as g=<sb2>/<sl2>, where sb is the mean square radius of gyration of the branched macromolecule in a given solvent, and sl is the mean square radius of gyration of an otherwise identical linear macromolecule in the same solvent at the same temperature. A value greater than 1 indicates an increased radius of gyration due to branching.

In some embodiments, the core polymer or PEG is a branched polymer that is capable of enhancing conjugation of the coating agent and core polymer. Exemplary branched polymers include 25 kDa branched polyethyleneimine (PEI) and 5 kDa branched methoxy-PEG.

### iv. Copolymers

In some embodiments, copolymers of PEG or other coating agents with any of the polymers described above may be used to make the MPPs. In some embodiments, the PEG or other coating agents may locate in the interior positions of the copolymer. Alternatively, the PEG or other coating agents may locate near or at the terminal positions of the copolymer.

In some embodiments, the nanoparticles are formed under conditions that allow regions of PEG or other coating agents to phase separate or otherwise locate to the surface of the particles. For example, the surface-localized PEG regions alone may perform the function of, or include, a surface-altering agent.

3. Particle Properties

As shown in the examples, the MPPs rapidly penetrate airway mucus at a greater rate of diffusivity than a reference nanoparticle, such as a mucus impermeable conventional particle (CP), e.g., uncoated PBAE particles formulated with PBAE only.

The rapid penetration of MPPs leads to widespread distribution and deep penetration in the mucus-covered lung airways in vivo, whereas CPs distribute as aggregates and primarily localized at mucosal surface lumen away from the airway epithelium.

The efficient mucus penetration of MPPs is essential for the particles to access to and subsequent uptake by pulmonary DC. For example, MPPs loaded with DNA vaccine components, such as antigen expressing DNAs and adjuvant, demonstrate enhanced delivery of inhaled DNA vaccine to pulmonary dendritic cells (DC). MPPs carrying both antigen expressing DNAs and adjuvant, preferably nucleic acid-based adjuvant, increase the percentage of DC positive for maturation markers (e.g. CD86⁺MHC-II⁺) significantly compared to carrier-free adjuvants and adjuvant-free counterpart (MPPs carrying only antigen expressing DNAs).

Inhaled MPPs carrying DNA vaccine components can be efficiently taken up by pulmonary DC resided in the lung interstitium and trafficked to the local lymph node, leading to robust and durable local and trans-mucosal immunity in lung and other remote mucosal surfaces compared to conventional systemic DNA vaccinations.

In addition, inhaled MPP-mediated vaccination provided greater systemic immunity than dose-matched antigen-expressing plasmids and adjuvants co-administered via routes commonly applied for systemic vaccination.

### i. Diffusivity

The transport rates of the MPPs can be measured using a variety of techniques in the art such as multiple particle tracking. Multiple particle tracking (MPT) measures various transport parameters such as mean squared displacements (MSD); MSD is a measure of the distances traveled by individual particles at a given time interval (i.e., timescale) and thus is directly proportional to particle diffusion rates (Schuster, et al., Adv. Drug Deliv. Rev., 91:70-91 (2015); Lai, et al., Methods Mol. Biol., 434:81-97 (2008); Suh, et al., Adv. Drug Deliv. Rev., 57:63-78 (2005)). In some embodiments, the rate of diffusion is measured by geometric ensemble MSD.

The MPPs may have a median MSD value of at least 0.1, 0.2, or 0.5 μm² (τ=1s) in a mucus sample. In some embodiments, the particles may diffuse through the pores of the airway mucus with a median MSD that is at least 5, 10, 20, 30, 50, 60, 80, 100, 125, 150, 200, 250, 500, 600, 750, 1000 or greater fold higher than a reference particle. In some embodiments, the particles may diffuse through the vaginal mucosa with a median MSD that is at least 5, 10, 20, 30, 50, 60, 80, 100, 125, 150, 200, 250, 500, 600, 750, 1000 or greater fold higher than a reference particle. In some embodiments, the particles may diffuse through the mucosal layer of GI tract with a median MSD that is at least 5, 10, 20, 30, 50, 60, 80, 100, 125, 150, 200, 250, 500, 600, 750, 1000 or greater fold higher than a reference particle.

The density of coating of PEG or other coating agents can affect the diffusion of nanoparticle within the airway mucus or other mucosal layers. In some embodiments, the median MSD at 1 sec of densely PEGylated particles in airway mucus is at least 0.1 μm² (τ=1s), for example, about 0.7 μm² (τ=1s). In some embodiments, the median MSD at 1 sec of densely PEGylated particles in airway mucus is at least 25-fold higher than that of non-PEGylated particles.

Nanoparticles exhibiting the average of median MSD values measured in multiple independent sputum/mucus samples to be equal to or greater than 0.1 (or log₁₀ MSD≥−1) are usual as mucus-penetrating nanoparticles. We might want to claim that the median MSD at 1 sec of densely PEGylated particles in airway mucus is at least 3-fold higher (0.1/0.03=3.33) than that of non-PEGylated particles

In this specific study, the average of median MSD values of mucus-penetrating and mucus-impermeable formulations are ˜0.7 and ˜0.03, respectively.

### ii. Size

In some embodiments, the MPPs have an average hydrodynamic diameter equal to or smaller than the pores in the mesh-like biological barrier, such as airway mucus. Particle size can be measured using any technique known in the art, for example using transmission electron microscopy or dynamic light scattering.

In another embodiment, the nanoparticles have an average diameter such that a majority of the nanoparticles do not become localized within cells or micro-domains within tissue compared to larger particles. The MPPs are highly stable in physiological solutions, such as bronchoalveolar lavage fluid (BALF) and artificial cerebrospinal fluid (aCSF). For example, the hydrodynamic diameter of the MPPs, upon incubation in a bronchoalveolar lavage fluid (i.e., physiological lung fluid), changes less than 25% within 4 hours.

As shown in the examples, MPPs formulated with both antigen expressing DNAs and adjuvants are the same to the MPPs formulated without adjuvants, e.g. the particle size, surface charges, and colloidal stability remain unchanged, demonstrating effective nucleic acids and adjuvant compaction. The effective compaction in MPPs provides protection of the nucleic acid payloads against extracellular nucleases.

### iii. Surface Charge

The presence of the PEG or coating agent can affect the zeta-potential of the particle. In some embodiments, the zeta potential of the particles is between −10 mV and 100 mV, between −10 mV and 50 mV, between −10 mV and 25 mV, between −5 mV and 20 mV, between −10 mV and 10 mV, between −10 mV and 5 mV, between −5 mV and 5 mV, or between −2 mV and 2 mV. For example, the MPPs can exhibit a zeta potential between −5 mV and less than 5 mV, measured in 10 mM NaCl at pH about 7. In preferred embodiments, the surface charge is near neutral.

### iv. Toxicity

The MPPs densely-coated with PEG or other coating agents are less toxic than non-coated particles. The in vitro or in vivo toxicity of particles can be assessed using any technique known in the art, such as histopathological assessment and BALF cell count. In some embodiments, the densely PEGylated biodegradable particles demonstrate significantly lower toxicity in vivo than non-PEGylated particles and similar safety profiles as clinically tested PEGylated particles.

B. Cargo

The particles typically include one or more immunogenic agent cargos encapsulated within, surrounded by, and/or distributed throughout the polymeric matrix of the particles. The particles can additionally or alternatively include additional cargos such as polypeptides, carbohydrates, small molecules, etc.

The methods are typically used to induce an immune response against an antigen in a subject in need thereof. Typically at least one cargo of the particles is an antigen, more preferably a nucleic acid encoding an antigen, or an adjuvant, preferably a nucleic acid adjuvant. In some embodiments, particles are co-loaded with both an antigen and an adjuvant.

1. Antigens

The particles are typically used to delivery an antigen, adjuvant, or combination thereof to a subject in need thereof.

Antigens can be peptides, proteins, polysaccharides, saccharides, lipids, nucleic acids, or combinations thereof. The antigen as generally used herein also encompasses nucleic acid (e.g., DNA or RNA) encoding all or part of one or more antigenic proteins or polypeptides. The DNA may be in the form of vector DNA such as plasmid DNA. Antigens may be provided as single antigens or may be provided in combination. Antigens may also be provided as complex mixtures of different nucleic acids encoding different polypeptide antigens, or even mixtures of polypeptides and nucleic acids. The antigen can be an immunogen.

In preferred embodiments, the particles are used to deliver components of a nucleic acid, typically DNA or RNA, vaccine. DNA-based vaccines are composed of purified closed-circular plasmid DNA or nonreplicating viral vectors containing genes that encode antigen (Vogel and Sarver, Clin Microbiol Rev., 8(3): 406-410 (1995)). RNA-based vaccines typically include an RNA, most typically an mRNA, encoding antigen. The use of mRNA as a vaccine vector obviates the potential safety issue of insertional mutagenesis related to DNA immunization.

### i. DNA Vectors

Typically, the particle are used to deliver one or more components of a nucleic acid vaccine. Nucleic acid vaccination is a technique for protecting against disease by injection with genetically engineered nucleic acids (typically DNA or RNA) so cells directly produce an antigen, producing a protective immunological response. Nucleic acid vaccines have potential advantages over conventional vaccines, including the ability to induce a wider range of immune response types.

DNA vaccine technology usually is based on bacterial plasmids that encode the polypeptide sequence of candidate antigens (Suschak, et al., Hum Vaccin Immunother. 13(12):2837-2848 (2017) doi: 10.1080/21645515.2017.1330236). The encoded antigen is typically expressed under a strong promoter active in eukaryotic cells and yielding high levels of transgene expression. Inclusion of transcriptional enhancers, such as Intron A, can enhance the rate of polyadenylation and nuclear transport of messenger RNA (mRNA).

Nucleic acid sequences encoding antigen is inserted into a plasmid backbone and then delivered to the host. Vaccine plasmid enters the nucleus of host myocytes and antigen presenting cells, where plasmid components are transcribed and antigenic protein is produced. The cell can provide endogenous post-translational modifications to antigens, producing native protein conformations. Vaccine-derived endogenous peptides are presented on MHC class I molecules. Engulfment of apoptotic or necrotic cells by APC also allows for cross-presentation of cell-associated exogenous antigens. Secreted antigen is captured and processed by APC, and presented on MHC class II. Antigen experienced APC migrate to the draining lymph node to stimulate CD4+ and CD8⁺ T cell populations. In addition, shed antigen can be captured by antigen-specific high affinity immunoglobulins on the B cell surface for presentation to CD4+ T cells, driving B cell responses.

A typical DNA vaccine vector includes genetic elements needed to drive intracellular expression of the foreign gene insert. These include one or more of: (i) a transcriptional promoter, (ii) an optional enhancer element to augment gene expression, (iii) the heterologous or foreign transgene encoding an antigenic gene product (e.g., a viral protein, etc.), and (iv) RNA-processing elements, primarily a polyadenylation signal and an optional intron element.

A marker gene (e.g., conferring antibiotic resistance) can be included for detection and/or selection. Plasmids may also contain bacterium-specific genetic sequences to allow large-scale production of the DNA, such as an antibiotic selectable marker, and a bacterial origin of replication to facilitate largescale amplification of the plasmid within this host cell.

Examples of suitable promoters, especially for the production of a DNA vaccine for humans, are known in the art, see, e.g., U.S. Published Application No. 2019/0022210, and include, but are not limited to, promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV) as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette included in the DNA molecule comprised by the attenuated strain of Salmonella of the present invention comprises the BGH polyadenylation site.

In addition to the regulatory elements required for expression of the heterologous antigen encoding gene, like a promoter and a polyadenylation signal, other elements can also be included in the recombinant DNA molecule. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are selected to be effective in the species to be immunized. The person skilled in the art can produce recombinant DNA molecules that are functional in a given subject species.

In some embodiments, small bacterial RNA-based antibiotic free selection markers are utilized. Noncoding RNA markers may be preferable to protein markers since proteins, like antibiotic resistance markers, can be expressed in the host organism after vector transfection, or horizontally transmitted to host bacteria. Noncoding RNA markers are also very small (<200 basepairs) which decreases the overall vector size; this is advantageous since vector transfection efficiency is inversely related to vector size.

DNA vaccine vectors with dramatically higher transgene expression, and can include a reduction in bacterial regions of the vector. For example, in minicircle vectors, the bacterial region is removed by the action of a phage recombinase during production, alleviated transgene silencing associated with large regions of bacterial. Alternatively, the vector can be a short bacterial region vector such as the Mini-Intronic Plasmid (MIP) and Nanoplasmid™ vector plasmid platforms. MIP vectors incorporate a RNA-OUT selection marker-pUC origin bacterial region within a 3′ UTR intron. In this configuration the bacterial region is within the transcription unit and the downstream polyA signal is linked to the eukaryotic promoter without an intervening selection marker or replication origin. Nanoplasmid™ vectors are RNA-OUT selection marker vectors in which the large pUC bacterial replication origin is replaced by a small R6K bacterial replication origin. In this configuration, the <500 basepair (bp) bacterial region separates the polyA signal and the eukaryotic promoter.

In particular embodiments, the DNA vaccine vector is a recombinant DNA molecule derived from or containing the same or similar elements as the commercially available pVAX1™ expression plasmid (Invitrogen, San Diego, Calif.). pVAX1™ is a plasmid vector for expression of proteins in eukaryotic cells which was specifically designed for use in the development of DNA vaccines by modifying the vector pcDNA3.1. Sequences not necessary for replication in bacteria or for expression of recombinant protein in mammalian cells were removed to limit DNA sequences with possible homology to the human genome and to minimize the possibility of chromosomal integration. Furthermore, the ampicillin resistance gene in pcDNA3.1 was replaced by the kanamycin resistance gene because aminoglycoside antibiotics are less likely to elicit allergic responses in humans.

The pVAX1™ vector contains the following elements: the human cytomegalovirus immediate-early (CMV) promoter for high-level expression in mammalian cells, the bovine growth hormone (BGH) polyadenylation signal for efficient transcription termination and polyadenylation of mRNA, and the kanamycin resistance gene as a selection marker. In addition pVAX1™ contains a multiple cloning site for insertion of the gene of interest as well as a T7 promoter/priming site upstream and a BGH reverse priming site downstream of the multiple cloning site to allow sequencing and in vitro translation of the clones gene. pVAX1™ expression vector was further modified by replacing the high copy pUC origin of replication by the low copy pMB1 origin of replication of pBR322. The low copy modification was made in order to reduce the metabolic burden and to render the construct more stable. The generated expression vector backbone was designated pVAX10. Importantly, data obtained from transfection experiments using the 293T human cell line demonstrated that the kanamycin resistance gene encoded on pVAX10 is not translated in human cells. The expression system thus complies with regulatory requirements.

DNA vaccine vectors can also be engineered to increase innate immune activation. DNA vaccines are potent triggers of innate immunity. Most of the intrinsic adjuvant effect of DNA is mediated by cytoplasmic innate immune receptors that nonspecifically recognize B DNA and activate Sting or Inflammasome mediated signaling, but unmethylated CpG sequences specific for TLR9 activation may also prime CD8 T cell responses. Thus, DNA vaccine vectors may be sequence modified to introduce immunostimulatory sequences (e.g., CG TLR9 agonists) into the vector. Additionally or alternatively the vector can encode immunostimulatory RNA, which can be designed to target endosome receptors, or cytoplasmic receptors such as RIG-I, MDAS and DDX3 are cytoplasmic. DNA vaccine vectors can also encode immunostimulatory sequences that selectively improve CTL responses to encoded antigen.

The vaccine plasmids can be produced in bacterial culture and purified for use with the particles.

### ii. Exemplary Antigens

Exemplary antigens are also provided. Any of the exemplary antigens herein can be encoded by a nucleic acid and form part of a nucleic acid vaccine. Thus, protein and polypeptide antigens, and nucleic acid sequence (e.g., transgenes and RNAs) encoding them are provided.

The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof.

Suitable antigens are known in the art and are available from commercial government and scientific sources. In one embodiment, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. The antigens can be recombinant polypeptides produced by expressing DNA encoding the polypeptide antigen in a heterologous expression system.

### a. Viral Antigens

A viral antigen can be isolated from any virus including, but not limited to, a virus from any of the following viral families Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### b. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

### c. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### d. Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeriaand Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### e. Cancer Antigens

A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

### 2. Molecular Adjuvants and Immunostimulatory Molecules

The particles can also be used to deliver one or more adjuvants and/or immunostimulatory molecules.

Most typically, the delivered adjuvants are molecular adjuvants. Molecular adjuvants include, for example, ligands for pattern recognition receptors, adaptor proteins, inflammation singling proteins, transcription factors, cytokines, chemokines, immune costimulatory molecules, toll-like receptor agonists or inhibitors of immune suppressive pathways, pathogen-recognition receptor (PRR) agonists, immune regulators (Li, et al., Curr Issues Mol Biol., 22:17-40 (2017) Epub 2016 Sep. 20. The adjuvants can be, for example, proteins or polypeptides, or nucleic acids encoding the same, including expression vectors. The adjuvants can also be nucleic acids such as oligonucleotides and inhibitory RNAs.

In some embodiments, the molecular adjuvant is an oligonucleotide that can serve as a ligand for pattern recognition receptors (PRRs). Examples of PRRs include the Toll-like family of signaling molecules that play a role in the initiation of innate immune responses and also influence the later and more antigen specific adaptive immune responses.

Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

In some embodiments, the oligonucleotide can serve as a ligand for a Toll-like family signaling molecule, such as Toll-Like Receptor 9 (TLR9). For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009)). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun 164(3):1617-2 (2000), each of which is incorporated herein by reference).

Other preferred PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDAS), which are best known as RNA-sensing receptors in the cytosol. Therefore, in some embodiments, the oligonucleotide contains a functional ligand for TLR3, TLR7, or RIG-I-like receptors, or combinations thereof.

Other adjuvants include Poly(I:C), a mismatched double-stranded RNA with one strand being a polymer of inosinic acid, the other a polymer of cytidylic acid, and variants thereof such as derivatives that have increased stability in body fluids (such as polyICLC), or reduced toxicity through reduced stability in body fluids (such as poly I:C12U).

Examples of suitable molecular adjuvant oligonucleotides, and methods of making them are known in the art, see for example, Bodera, P. Recent Pat Inflamm Allergy Drug Discov. 5(1):87-93 (2011), and Li and Petrovsky, Current Issues in Molecular Biology, 22:17-40 (2016).

In some embodiments, the oligonucleotide cargo includes two or more immunostimulatory sequences.

The oligonucleotide can be between 2-100 nucleotide bases in length, including for example, 5 nucleotide bases in length, 10 nucleotide bases in length, 15 nucleotide bases in length, 20 nucleotide bases in length, 25 nucleotide bases in length, 30 nucleotide bases in length, 35 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

Adjuvants and immunostimulator agents also include receptor ligands, proteins, cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor, or nucleic acids encoding the same.

In some embodiments, tumor suppressor genes, such as p53 and Rb can be complexed into particles to be used for cancer patients.

Any of the adjuvants and/or immunomodulatory agents can be dispersed in the particles or be covalently or non-covalently attached to one or more of the polymeric components of the particles.

### 3. Additional Active Agents

Other therapeutic, prophylactic, and/or diagnostic agents can be co-delivered depending on the application. The additional active agents can be non-nucleic acid active agents. The additional agents can be dispersed in the MPPs or be covalently or non-covalently attached to one or more polymeric components of the MPPs.

Suitable additional active agents include, but are not limited to, other nucleic acid-based medicine, anti-inflammatory drugs, antiproliferatives, chemotherapeutics, vasodilators, and anti-infective agents. In some embodiments, the MPPs contain one or more antibiotics, such as tobramycin, colistin, or aztreonam. The nucleic acid delivery MPPs can optionally contain one or more antibiotics which are known to possess anti-inflammatory activity, such as erythromycin, azithromycin, or clarithromycin. Particles may also be used for the delivery of chemotherapeutic agents, and anti-proliferative agents.

### III. Methods of Making the Nanoparticles

Methods for formulating sub-100 nm, compact, colloidally stable PBAE MPPs that have a dense surface coverage of a hydrophilic and neutrally charged polymer (i.e. PEG) are disclosed. The formulation methods are highly tailorable and thus can be applied to various biodegradable cationic polymers.

The nanoparticles were formulated with the following size range

MPP—mucus penetrating nanoparticles

CP— conventional formulation without PEG coating

| | Hydrodynamic | | |
|---|---|---|---|
| | Diameter ± | | ζ-potential ± |
| Particle Type | SEM, nm | PDI | SEM, mV |
| pOVA-MPP | 55 ± 1 | 0.1 | 1.6 ± 0.3 |
| CpG/pOVA-MPP | 54 ± 1 | 0.1 | 1.7 ± 0.2 |
| p(I:C)/pOVA-MPP | 57 ± 1 | 0.1 | 2.2 ± 0.1 |
| pOVA-CP | 120 ± 4 | 0.1 | 32 ± 2 |

^{†}Hydrodynamic diameter and PDI were measured by dynamic light scattering in water (pH 7.0). Data represent the mean ± SEM (n ≥ 3).
^{‡}ζ-potential was measured by laser Doppler anemometry in 10 mM NaCl (pH 7.0). Data represent the mean ± SEM (n ≥ 3).

As previously reported, PEGylation of cationic polymers may have negative influences on DNA complexation due to reduction of available positive charges resulting from the PEG conjugation to the amine groups of cationic polymers and additional steric hindrance imposed by grafted PEG chains. The technique of incorporating a non-PEGylated polymer core to allow compact DNA complexation was used to overcome this limitation and achieve dense PEG surface coating. Achieving effective DNA compaction using a mixture of PEGylated and non-PEGylated PBAE required thorough characterization and careful optimization of formulation parameters including but not limited to free polymer/DNA weight ratio, DNA/adjuvant weight ratio, PBAE/PBAE-PEG ratio, pH of DNA, and polymer solutions, type of buffering solution and method of mixing.

The formulation methods can be applied to various biodegradable cationic polymers and hydrophilic and neutrally charged polymers.

A. Polymer Preparation

The polymers can be synthesized using known methods or purchased. PEG or other coating agents can be conjugated to the biodegradable core polymer using a variety of techniques known in the art depending on whether the coating is covalently or non-covalently associated with the particles.

In some embodiments, the PEG or other coating agent can be covalently attached to the core polymer by reacting functional groups on the particles with reactive functional groups on the PEG or other coating agent to make a copolymer. For example, PEG-succinimidyl succinate can be reacted with primary amine groups to covalently attach the coating agent via an amide bond.

In some embodiments, polyethylene glycol (PEG)-conjugated poly(β-amino ester) (PBAE) (PBAE-PEG) polymer is synthesized by a two-step reaction from the uncapped base PBAE polymers: end diacrylate group capping and purification can be conducted using with 1,3-diaminopropane; subsequently, the end capped PBAE polymers and 2.05 molar excess of 5 kDa methoxy-PEG-N-hydroxysuccinimide can be mixed, vacuumed and purged with nitrogen. The extent of PEGylation of the resulting PBAE copolymer can be varied by varying the molar ratio of PEG added to the PBAE.

B. MPPs Formulation

The MPPs can be formed from one or more cationic polymers, one or more PEGs or other coating agents, and cargo using any suitable method for the formation of polymeric particles known in the art.

Methods of making MPPs densely coated with PEG that are optimized for the delivery of nucleic acids across biological barriers are provided. The cargo typically includes nucleic acids encoding a polypeptide antigen or the polypeptide antigen, and/or adjuvants. For example, formulations of MPPs for delivery of DNA vaccine components such as antigen expressing nucleic acids and adjuvants for pulmonary administration are disclosed.

Factors that can influence the physicochemical properties of the nanoparticles can include: the mass ratio of free polymer and conjugated polymer within the blended polymer; the mass ratio of cargo to blended polymer; the mass ratio of nucleic acid to adjuvant; the volume ratio of cargo added to the blended polymer; the rate at which cargo and blended polymer are combined, and the concentration ratio of the cargo to the blended polymer.

1. Blended Polymer

In some embodiments, nanoparticles are formed of a mixture of PEGylated and non-Pegylated biodegradable polymers, such as charged biodegradable polymers. The blended polymer containing free (i.e. non-PEGylated) and conjugated (i.e. PEGylated) biodegradable polymers can retain a charge that is useful for enhancing compaction with nucleic acid, as compared to polymers that contain 100% PEG-conjugated biodegradable polymer, or 100% free biodegradable polymer. In some embodiments, the use of a free biodegradable polymer/PEG-conjugated biodegradable polymer blend enables formation of a compact nanoparticle that has a smaller hydrodynamic radius and is more stable than a reference particle. The non-PEGylated biodegradable polymers can contribute a defined amount of the total free amines, such as 5%, 10%, 15%, 20%, 25%, 30%, 35%, 40%, 45%, 50% or more than 50% of the total free amines in the particles. In some embodiments, the ratio of non-PEGylated biodegradable polymers to PEG-conjugated biodegradable polymers is optimized for colloidally stable nanoparticles with a diameter less than 100 nm and a near neutral surface charge. The blended polymer can contain a mass ratio of free biodegradable polymer to biodegradable polymer conjugated with PEG between 0.5 and 1, or more than 1, based on the mass of the biodegradable polymer. In a particular embodiment, the blended polymer contains a mass ratio of free biodegradable polymer to biodegradable polymer conjugated with PEG of about 0.67, based on the mass of the biodegradable polymer.

### 2. Nucleic Acids-Loaded MPPs Formulation

In some embodiments, MPPs are formed using a mass:mass ratio of the blended polymer to cargo that is optimized for producing colloidally stable nanoparticles with a diameter less than 100 nm and a near neutral surface charge. In preferred embodiments, the MPPs contain both antigen expressing nucleic acids and adjuvants such as nucleic acid-based adjuvants. Typically, the mass:mass ratio of the biodegradable polymer in the blended polymer to the total of antigen expressing nucleic acids and nucleic acid-based adjuvants (e.g. PBAE:nucleic acid) is up to 1,000:1, such as 500 to 1, 100 to 1, or less than 100 to 1, such as 60:1. In some embodiments, the mass:mass ratio of the antigen expressing nucleic acid to the adjuvants (i.e. nucleic acid-based adjuvant) is up to 10:1, such as 8:1, 5:1, or 4:1.

In further embodiments, MPPs are formed using a volume:volume ratio of cargo solution added to blended polymer solution that is optimized for producing colloidally stable nanoparticles with a diameter less than 100 nm and a near neutral surface charge. In some embodiments, up to 10 volumes of cargo are added to one volume of blended polymer. In preferred embodiments, 5 volumes of cargo solution is added to one volume of blended polymer solution, where the cargo solution includes both antigen expressing nucleic acids and nucleic acid-based adjuvants.

The rate at which the cargo is added to the blended polymer solution can also influence the physicochemical properties of the MPPs. In some embodiments, the cargo is added to the blended polymer at a steady rate of up to 10 ml/min, for example, the cargo is added to the blended polymer at a rate of about 1 ml/min Preferably, the cargo solution including antigen expressing nucleic acids and nucleic acid-based adjuvants, is added to the blended polymer at about 0.1 ml/min.

The concentration of the blended polymer can be up to 2,000 times the concentration of the nucleic acid, such as up to 300 times. In some embodiments, the concentration of the blended polymer is about 100 mg/ml and the concentration of the nucleic acid is about 0.1 mg/ml.

The concentration of the nucleic acid solution that can be used is 0.01, 0.05, 0.1, 0.2 or greater than 0.2 mg/ml up to 1 mg/ml. 0.1 mg/ml concentration of nucleic acid is preferred.

In circumstances where a monodisperse population of particles is desired, the particles may be formed using a method which produces a monodisperse population of nanoparticles. Alternatively, methods producing polydisperse nanoparticle distributions can be used, and the particles can be separated using methods known in the art, such as sieving, following particle formation to provide a population of particles having the desired average particle size and particle size distribution.

Methods of making polymeric particles are known in the art. Common microencapsulation techniques include, but are not limited to, spray drying, interfacial polymerization, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

Pharmaceutically acceptable excipients, including pH modifying agents, disintegrants, preservatives, and antioxidants, can optionally be incorporated into the particles during particle formation. As described above, one or more additional active agents can also be incorporated into the MPPs during particle formation.

### i. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

### ii. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

### iii. Solvent Evaporation Microencapsulation

Methods for forming microspheres using solvent evaporation techniques are described in E. Mathiowitz, et al., J. Scanning Microscopy, 4:329 (1990); L. R. Beck et al., Fertil. Steril., 31:545 (1979); L. R. Beck, et al., Am. J Obstet. Gynecol., 135(3) (1979); S. Benita, et al., J. Pharm. Sci., 73:1721 (1984); and U.S. Pat. No. 3,960,757 to Morishita, et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

### iv. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

### v. Microfluidics

Nanoparticles can be prepared using microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution.

Other methods known in the art that can be used to prepare nanoparticles include, but are not limited to, polyelectrolyte condensation (see Suk, et al., Biomaterials, 27, 5143-5150 (2006)); single and double emulsion (probe sonication); nanoparticle molding, and electrostatic self-assembly (e.g., polyethylene imine-DNA or liposomes).

### IV. Formulations

The nanoparticles can be administered in combination with a physiologically or pharmaceutically acceptable carrier, excipient, or stabilizer. The term “pharmaceutically acceptable” means a non-toxic material that does not interfere with the effectiveness of the biological activity of the active ingredients. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. For example, the particles can be formulated in sterile saline or buffered solution for injection into the tissues or cells to be treated. The particles can be stored lyophilized in single use vials for rehydration immediately before use. Other means for rehydration and administration are known to those skilled in the art.

Preferably, the compositions are formulated with an effective amount of nanoparticle carriers in a pharmaceutical carrier appropriate for administration to a mucosal surface. Pharmaceutical formulations and methods for the pulmonary administration of active agents to patients are known in the art. Pharmaceutical formulations can be administered to any mucosal surface in a patient to treat or lessen one or more symptoms. Generally, the formulations are administered to the pulmonary tract. Aerosolized pharmaceutical formulations can be delivered to the lungs, preferably using a device, such as a dry powder inhaler, nebulizer, or pressurized metered dose inhaler (pMDI). Liquid formulations can also be administered to the respiratory tract by other suitable methods such as intranasal instillation, intratracheal instillation, and intratracheal injection. The formulations can also be administered to other mucosal surfaces including nasal, buccal, rectal and vaginal surfaces.

The respiratory tract is the structure involved in the exchange of gases between the atmosphere and the blood stream. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung, where the exchange of gases occurs.

Formulations can be divided into dry powder formulations and liquid formulations. Both dry powder and liquid formulations can be used to form aerosol formulations. The term aerosol as used herein refers to any preparation of a fine mist of particles, which can be in solution or a suspension, whether or not it is produced using a propellant.

The nanoparticles are typically formulated as an immunogenic compositions or as components in vaccines. Typically, immunogenic compositions herein include an adjuvant, an antigen, or a combination thereof. When administered to a subject in combination, the adjuvant and antigen can be administered in separate pharmaceutical compositions, or they can be administered together in the same pharmaceutical composition.

One or both of the antigen and the adjuvant can be loaded into or onto MPP. Typically, the antigen (e.g., a nucleic acid encoding polypeptide), is loaded into or onto MPP. Adjuvant (e.g., a molecular adjuvant) can also be loaded into or onto the same or different MPP. Thus, in some embodiments, antigen and adjuvant are co-loaded.

As demonstrated in the example, nanoparticles can be co-loaded with antigen-encoding plasmids.

In addition or alternative to the MPP-loaded adjuvants discussed above, the compositions and methods may also include separate non-MPP delivery of one or more adjuvants from above, or another adjuvant. Other adjuvants include, but are not limited to, alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

The adjuvant can also be oil emulsions (e.g., Freund's adjuvant); saponin formulations; virosomes and viral-like particles; bacterial and microbial derivatives; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol). Adjuvants are loaded into the core of the particle and thus do not interfere with mucus-penetrating property.

### V. Methods of Use

The MPPs provide an effective, biocompatible, and non-toxic vehicle for the delivery of nucleic acids to the cells of a subject. The MPPs are readily taken up by many cell types and efficiently deliver nucleic acids to biological targets.

The uniquely high mobility of the MPPs in mucus facilitates cross-sectional penetration and lateral spread of the particles through mucus such as the airway mucus gel layer, thereby enhancing the probability of particle encounter and uptake by cells reside in mucosa such as pulmonary DC. The enhanced MPP uptake by cells is also because of their ability to circumvent the physiological mucociliary clearance (MCC).

For example, the experiments below show that the MPPs effectively delivered DNA vaccine components including both antigen expressing DNAs and nuclei acid-based adjuvants to the pulmonary DC of a subject following intratracheal administration and mediated significantly stronger and more durable adaptive immunity in the lung and other remote mucosal surfaces compared to conventional systemic DNA vaccination. In addition, inhaled MPP-mediated vaccination provided greater systemic immunity than dose-matched antigen-expressing plasmids and adjuvants co-administered via routes commonly applied for systemic vaccination.

Inhaled MPPs carrying DNA vaccine components are efficiently taken up by pulmonary DC resided in the lung interstitum, trafficked to the local lymph node, and subsequently elicited strong pulmonary immunity. Inhaled reference particles such as PBAE CPs (particles formulated with PBAE only) showed negligible DC uptake despite its superior in vitro DC uptake capacity compared to MPPs. The intrinsic ability to mediate DC uptake (i.e. in vitro DC uptake) alone is insufficient in promoting particle uptake by pulmonary DC in vivo. This showed that the immune response could be enhanced.

A. Methods of Increasing an Immune Response

In some embodiments, the MPPs can deliver nucleic acids and/or exogenous proteins to a subject to stimulate desired immune responses in the subject. The delivery of antigen expressing nucleic acids or antigens via the MPPs confers protective immunity to infectious agents such as viruses and bacteria.

Methods for DNA vaccination using antigen expressing DNAs and adjuvants within MPPs are provided. In particular, MPPs-mediated vaccination is administered to the pulmonary system and mediates strong and durable immunity not only at the site of administration (i.e. lung), but also other remote mucosal surfaces such as GI and vaginal tracts and systemically in the spleen.

The methods typically include administering a subject in need thereof one or more immunogenic compositions including particles loaded with antigen, adjuvant, or a combination thereof in an effective amount to induce, increase or enhance an immune response. The “immune response” refers to responses that induce, increase, or perpetuate the activation or efficiency of innate or adaptive immunity. The particles can be delivered parenterally (by subcutaneous, intradermal, or intramuscular injection) through the lymphatics, or by systemic administration through the circulatory system, though the most preferred method of delivery is topically to a mucosal surface.

In some embodiments, the same or different particle compositions are administered in multiple doses at the same or various locations throughout the body.

The immune response can be induced, increased, or enhanced by the composition compared to a control, for example an immune response in a subject induced, increased, or enhanced by the cargo alone, or the cargo delivered using an alternative delivery strategy (e.g., non-MPP particles). In some embodiments, the composition reduces inactivation and/or prolongs activation of T cells (i.e., increase antigen-specific proliferation of T cells, enhance cytokine production by T cells, stimulate differentiation and effector functions of T cells and/or promote T cell survival) or overcome T cell exhaustion and/or energy.

The compositions can be used, for example, to induce an immune response, when administering the cargo alone, or the cargo in combination with an alternative delivery system, is ineffectual. The compositions can also be used to enhance or improve the immune response compared to administering cargo alone. In some embodiments, the compositions may reduce the dosage required to induce, increase, or enhance an immune response; or reduce the time needed for the immune system to respond following administration.

Cargo-loaded particles can be used as prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a viral antigen in a subject infected with a virus, or cancer antigen in a subject with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

In some embodiments, one or more immunogenic compositions including MPP-loaded antigen, e.g., DNA vaccine-based antigen, alone or preferably in combination with adjuvant, e.g., MPP-loaded molecular adjuvant is administered to a subject in an effective amount to increase antigen uptake in pulmonary DC (CD11⁺CD170⁻); increase DC maturation (e.g., percentage of DC positive for maturation markers (CD86⁺MHC-II⁺)); increase DC number or frequency, particularly pulmonary DCs (CD11c⁺CD170⁻) in the lung airway interstitium; increase DC migration to the lymph nodes (e.g., mediastinal LN); increase antigen-specific CTL response (e.g., effector T166 cells (CD3_{ε}⁺ CD8⁺) positive for antigen), particularly in the lung, mediastinal LN and/or spleen; increase activated CD8⁺ T-cells (IFN-g⁺CD8⁺) and/or increase frequencies of CD4⁺ T-cell activation (e.g., percentage of IFN-g⁺CD4⁺ T-cells), particularly in the lung, mediastinal LN and/or spleen; increase dissemination of antigen-specific CD8⁺ T cells to, and/or CTL responses in, tissues distal to the site of administration; increase antigen specific T-cell memory biased towards the effector memory phenotype both at the site of administration (i.e., lung) and/or systemically in the spleen, preferably wherein the bias is most prominent in the lung (e.g., greater frequency of T_{EM} (CD44^{hi}CD62L^{lo} compared to that of T_{CM} (CD44^{hi}CD62L^{hi}); increases gut homing integrin (alpha4beta7) in the CD8+ T cells in mediastinal lymph node; or a combination thereof.

In some embodiments, the composition induces an improved effector cell response, such as a CD4+ or CD8⁺ T-cell immune response, against at least one of the component antigen(s) or antigenic composition compared to the effector cell response obtained with the corresponding composition without delivery using MMP. The term “improved effector cell response” refers to a higher effector cell response such as a CD8 or CD4 response obtained in a human patient after administration of the composition than that obtained after administration of the same composition without MMP-based delivery. In some embodiments, the improved effector cell response present in one or more of the lungs, lymph nodes, or spleen.

The improved effector cell response can be obtained in an immunologically unprimed patient, i.e. a patient who is seronegative to the antigen. This seronegativity may be the result of the patient having never faced the antigen (so-called “naïve” patient) or, alternatively, having failed to respond to the antigen once encountered. In some embodiments, the improved effector cell response is obtained in an immunocompromised subject.

In some embodiments, immunogenic composition increases the primary immune response as well as the CD8 response. Thus, the composition can induces an improved CD4 T-cell. This method may allow for inducing a CD4 T cell response which is more persistent in time. Preferably the CD4 T-cell immune response, such as the improved CD4 T-cell immune response obtained in an unprimed subject, involves the induction of a cross-reactive CD4 T helper response. In particular, the amount of cross-reactive CD4 T cells is increased. The term “cross-reactive” CD4 response refers to CD4 T-cell targeting shared epitopes for example between influenza strains.

In a preferred embodiment, the composition increases the number of T cells producing IFN-gamma, TNF-alpha, or a combination thereof, or increases the production of IFN-gamma, TNF-alpha, or a combination thereof in the existing T cells.

In some embodiments, the administration of the immunogenic composition alternatively or additionally induces an improved B-memory cell response in patients compared to a control. An improved B-memory cell response is intended to mean an increased frequency of peripheral blood B lymphocytes capable of differentiation into antibody-secreting plasma cells upon antigen encounter. Such a result can be measured, for example, by stimulation of in vitro differentiation.

B. Methods of T Cell Therapy

In some embodiments, antigen-specific cytotoxic effector CD8⁺ T cells are primed by the composition and administered to a subject in need thereof. The T cells can be harvested from a treated subject, and optionally expanded in culture, or primed and expanded in vitro.

For example, in a particular embodiment, the method is one of adaptive T cell therapy. Methods of adoptive T cell therapy are known in the art and used in clinical practice. Generally adoptive T cell therapy involves the isolation and ex vivo expansion of tumor specific T cells to achieve greater number of T cells than what could be obtained by vaccination alone. The tumor specific T cells are then infused into patients (e.g., with cancer) in an attempt to give their immune system the ability to overwhelm remaining tumor or infection via T cells which can attack and kill the target. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood after they have received treatment or immunization with the composition. Methods of priming and activating T cells in vitro for adaptive T cell therapy are known in the art. See, for example, Wang, et al., Blood, 109(11):4865-4872 (2007) and Hervas-Stubbs, et al., J. Immunol., 189(7):3299-310 (2012). The methods can be used in conjunction with the composition to prime and activate T cells against a target such as cancer or infection.

Historically, adoptive T cell therapy strategies have largely focused on the infusion of tumor antigen specific cytotoxic T cells (CTL) which can directly kill tumor cells. However, CD4+T helper (Th) cells can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th1 have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

C. Diseases to Be Treated

1. Cancer

The immunogenic compositions are useful for stimulating or enhancing an immune response in host for treating cancer. Thus, method of treating cancer also provided the types of cancer that may be treated with the provided compositions and methods include, but are not limited to, the following: bladder, brain, breast, cervical, colo-rectal, esophageal, kidney, liver, lung, nasopharyngeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions can be administered in as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the compositions may reduce tumor size, or slow tumor growth compared to a control. The stimulation of an immune response against a cancer may be coupled with surgical, chemotherapeutic, radiologic, hormonal and other immunologic approaches in order to affect treatment.

In some embodiments, the cancer is a lung cancer. Exemplary lung cancers include, but are not limited to, small cell lung cancers (SCLC) and non-small cell lung cancers (NSCLC) including adenocarcinoms, squamous cell carcinomas, and large cell carcinomas, as well as bronchial carcinoids, cancers of supporting lung tissue such as smooth muscle, blood vessels, or cells involved in the immune response, and metastatic cancers from other primary tumors in the body.

2. Infectious Diseases

The compositions are also useful for treating acute or chronic infectious diseases. Because viral infections are cleared primarily by T-cells, an increase in T-cell activity is therapeutically useful in situations where more rapid or thorough clearance of an infective viral agent would be beneficial to an animal or human subject. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the MMP-loaded particles can be administered topically to treat viral skin diseases such as herpes lesions or shingles, or genital warts. The compositions can also be administered to treat systemic viral diseases, including, but not limited to, AIDS, influenza, the common cold, or encephalitis.

Representative infections that can be treated, include but are not limited to infections cause by bacteria, fungi and parasites, such as Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiment, the type of disease to be treated or prevented is a chronic infectious disease caused by a bacterium, virus, protozoan, helminth, or other microbial pathogen that enters intracellularly and is attacked, e.g., by cytotoxic T lymphocytes.

In some embodiments, the infection is a lung infection. Lung infections including, but are not limited to, upper respiratory infections such as the common cold, sinusitis, pharyngitis, epiglottitis and laryngotracheitis; and lower respiratory infections such as bronchitis, bronchiolitis and pneumonia.

D. Administration

The immunogenic compositions can be administered by a variety of routes of administration. In certain embodiments, the compositions are administered directly to the pulmonary system. In other embodiments, the compositions are administered systemically.

In general the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

Regardless of systemic, pulmonary, intrathecal, or intravaginal administration, etc., penetration of cargo agents in the mucus and other tissues has been a key hurdle to effective therapy and diagnostics. For example, numerous studies using viral, nanoparticle, and convection-enhanced delivery have failed due to limited movement of substances within the airway mucus. Therefore, defining the critical limiting parameters and designing strategies to enhance mucosal penetration will likely improve the efficacy of these treatments. Densely-pegylated nanoparticles offer numerous additional advantages, including increased particle diffusion, improved stability, and prolonged sustained-release kinetics. These factors are known to correlate with the efficacy of many therapeutics and will likely have a significant impact on the utility of nano-sized carriers for diagnostic and therapeutic delivery to cells reside in mucosa such as DC reside in the airway mucosa.

The present invention will be further understood by reference to the following non-limiting examples.

### EXAMPLES

### Example 1. Mucus Penetration Enhances Uptake of DNA-Loaded Nanoparticles by Pulmonary Dendritic Cells

Materials and Methods

Polymer Synthesis

Poly(β-amino ester) (PBAE) polymers were synthesized using the a method as detailed below (Mastorakos, et al., Proc Natl Acad Sci USA, 112: 8720-8725 (2015)). Briefly, a two-step Michael addition reaction was used to synthesize non-PEGylated PBAE polymers. First, 4-amino-1 butanol and 1,4-butanediol diacrylate were reacted at a 1.1:1 molar ratio at 90° C. for 16 h in tetrahydrofuran (THF) to yield acrylate-terminated PBAE polymers possessing molecular weight (MW) of 6.0±0.2 kDa. The synthesized polymers were purified by washing three times with cold ether and dried under vacuum without exposure to light for 14 days to remove residual ether. The acrylate-terminated PBAE polymers were then reacted with 30-molar equivalents of 2-(3-aminopropylamino) ethanol in THF at room temperature for 6 h, followed by the purification and solvent-removal steps.

For preparing PEG-PBAE polymers, non-PEGylated PBAE polymers were first synthesized using the aforementioned method with some modifications. Briefly, 4-amino-1 butanol and 1,4-butanediol diacrylate were reacted at a 1.2:1 molar ratio to yield acrylate terminated PBAE polymers possessing MW of 4.0±0.2 kD 329 a and subsequently reacted with 30 molar equivalents of 1,3-diaminopropane. These intermediate polymers were extensively washed and dried as described above after each reaction step was completed. The polymers were then reacted with 2.05 molar equivalents of methoxy-PEG-succinimidyl succinate (JenKem) in THF overnight at room temperature, followed by purification and solvent-removal steps, to yield the final product of PEG-PBAE polymers. Both non-PEGylated PBAE and PEG-PBAE polymers were dissolve in dimethyl sulfoxide and stored at −20° C. for future use.

¹H Nuclear Magnetic Resonance (NMR) Spectroscopy

Polymers were characterized by NMR as previously detailed (Mastorakos, et al., Proc Natl Acad Sci USA, 112: 8720-8725 (2015)). Briefly, ¹H NMR spectra of non-PEGylated PBAE and PEG-PBAE dissolved in deuterated methanol (MeOH-d4; Cambridge Isotope Laboratories) were recorded on a Bruker spectrometer (500 MHz). 1H chemical shifts were reported in ppm (δ) and the MeOH peak was used as an internal standard. Data were processed using iNMR software.

DNA-Loaded Nanoparticle Formulation & Characterization

The OVA-expressing plasmid used in this study (pCI-neo-OVA) was a gift from Dr. Maria Castro (Addgene plasmid #25098; (Yang, et al., Proc. Natl. Acad. Sci. USA, 107:4716-4721 (2010)). The nucleic acid-based adjuvants, including CpG and poly(I:C), were purchased from InvivoGen. For microscopic and flow cytometric analysis, plasmid DNA was fluorescently labeled with the either Cy5 or MFP488 fluorophores using the Mirus Label IT tracker intracellular nucleic acid localization kit (Mirus Bio) according to the manufacturer's instruction.

DNA-loaded nanoparticles was formulated as detailed below (Mastorakos, et al., Proc Natl Acad Sci USA, 112: 8720-8725 (2015)). The polymer solution was prepared with PBAE only or a mixture of PBAE and PBAE-PEG (at a wt/wt ratio of 2:3 based on PBAE mass) for CP or MPP, respectively. To engineer DNA-loaded nanoparticles, five volumes of nucleic acids, including labeled or unlabeled plasmid DNA at 0.1 mg/mL were added dropwise to one volume of a polymer solution at a PBAE-to-nucleic acid wt/wt ratio of 60:1 while vortexing. DNA-loaded nanoparticles were then washed with five volumes of ultrapure distilled water at 950×g for 8 minutes each time and concentrated to 0.5 mg/mL using Amicon Ultra Centrifugal Filters (100,000 molecular-weight cutoff; Millipore).

For the nanoparticle characterization, hydrodynamic diameters and polydispersity index were measured in ultrapure water by dynamic light scattering and ζ-potential was measured in 10 mM NaCl at pH 7.0 by laser doppler anemometry, using a Zetasizer Nano ZS90 (Malvern Instruments). Transmission electron microscopy (H7600; Hitachi High Technologies America) was conducted to determine the morphology and geometric dimension of DNA-loaded nanoparticles.

Cell Culture

Bone marrow-derived dendritic cells (JAWSII) cells were purchased from ATCC and SIINFEKL (SEQ ID NO:1) expressing LLC cells were kindly provided by Dr. Amer A. Beg (Moffitt Cancer Center). JAWSII cells were maintained in Alpha Minimum Essential Medium (MEM) supplemented with ribonucleosides, deoxyribonucleosides, 4 mM L-glutamine (Thermo Fisher Scientific), 1 mM sodium pyruvate, 5 ng/ml murine granulocyte-macrophage colony-stimulating factor (Thermo Fisher Scientific), 20% HI-FBS (Thermo Fisher Scientific) and 1% penicillin/streptomycin. SIINFEKL (SEQ ID NO:1)-expressing LLC cells were cultured in MEM supplemented with 10% HI-FBS and 1% penicillin/streptomycin.

In Vitro Dendritic Cell Uptake and Activation Studies

JAWSII cells were plated on a 12-well plate at 200,000 cells/well for both studies. To evaluate DC uptake, the cells were treated with either MPP or CP carrying 0.5 μg of Cy5-labeled pOVA. Cells in controls groups were treated with PBS or carrier-free nucleic acids at a same dose. Cells were incubated at 37° C. for 6 hours prior to flow cytometric analysis.

Animal Studies

All animals were handled in accordance with the policies and guidelines of the Johns Hopkins University Animal Care and Use Committee. Female C57BL/6 mice (6-8 week old; Charles River) were anesthetized with an intraperitoneal injection of 2,2,2-tribromoethanol (Sigma-Aldrich) or isoflurane. To evaluate in vivo performances of MPP (e.g. IT CpG/pOVA-MPP group), mice were treated with a single intratracheal dose of MPP carrying 20 μg of either fluorescently labeled or unlabeled pOVA with or without 4 μg of CpG at a volume of 50 μL via a microsprayer (MicroSprayer Aerosolizer Model IA-1C; Penn-Century). For comparison, mice in IT CpG/pOVA group were identically treated with carrier-free nucleic acids at the same dose and volume. In parallel, mice in ID and IM-EP CpG/pOVA groups received a mixture of 20 μg pOVA and 4 μg CpG at a volume of 24 μL in footpad and quadriceps, respectively, using a 30G needle attached to a gas-tight syringe (Hamilton). For the IM-EP CpG/pOVA group, an additional procedure of electroporation (Harvard Apparatus ECM830) was conducted at 2×60 ms pulses and 200 V/cm.

To evaluate the uptake of DNA-loaded nanoparticles by pulmonary DC in vivo, mice were intratracheally treated with either of CP or MPP carrying MFP488-labeled pOVA and sacrificed 16 hours after the administration to determine the uptake using flow cytometric analysis. For microscopic observation of DNA-loaded nanoparticles, lung tissues were harvested different time points after the intratracheal administration, embedded in optimum cutting temperature compound (Finetek) solution, cryosectioned using a CM1950 cryostat (Leica Biosystems) and imaged using a confocal LSM 710 microscope (Carl Zeiss). Specifically, the distribution of DNA-loaded nanoparticles in tracheal lumen and lung airway interstitium was determined 1 hour after mice received either of CP or MPP carrying Cy5-labeled pOVA with and without CpG, respectively. In parallel, trafficking of DNA-loaded nanoparticles to the mediastinal LN and lung was evaluated 48 hour after treatment with MPP carrying Cy5-labeled pOVA and CpG. Cells were labeled or stained using antibodies against CD11c (abcam) and/or CD170 (abcam), Alexa Fluor 568 Goat Anti-Armenian hamster IgG secondary antibody (abcam), Alexa Fluor 488 Goat Anti-Rabbit IgG secondary antibody (abcam) and 4′,6-diamidino-2-phenylindole (DAPI). Of note, microscopic settings were carefully adjusted to avoid introduction of any background fluorescence using lung tissues sections from untreated mice.

Statistical Analysis

Statistical analyses of two or multiple comparisons were conducted using Student's t-test or one way analysis of variance (ANOVA), respectively, and survival of animals was compared using the log rank test in Graphpad Prism 7. Differences were considered to be statistically significant at a level of p<0.05.

Results

Nanoparticles capable of efficiently penetrating airway mucus for inhaled delivery of model antigen-expressing plasmids were engineered using a blend method (Mastorakos, et al., Proc Natl Acad Sci USA, 112: 8720-8725 (2015)). Briefly, a mixture of poly(β-amino ester) (PBAE) and polyethylene glycol (PEG)-conjugated PBAE (PEG-PBAE) at an optimized ratio was used to compact ovalbumin (OVA)-expressing plasmids (pOVA) to yield mucus-penetrating particles (pOVA-MPP). In parallel, mucus impermeable conventional particles carrying pOVA (pOVA-CP) were formulated with PBAE only. The pOVA-MPP exhibited small particle diameters of 55±1 nm and near-neutral surface charges of 1.6±0.3 mV (Table 1), physicochemical properties that render particles muco-inert and permeable to airway mucus (Kim, et al., J. Control. Release, 240:465-488 (2016); Suk, et al., Adv. Drug Deliv. Rev., 99:28-51 (2016)). In contrast, pOVA-CP possessed larger particle diameters of 120±4 nm and highly positive, muco-adhesive, surface charges of 32±2 mV (Table 1). Widespread distribution and deep penetration of pOVA-MPP in the mucus-covered lung airways in vivo were observed 1 hour after the intratracheal administration. Identically administered pOVA-CP were sparsely distributed as aggregates and primarily localized at mucosal surface lumen away from the airway epithelium (observed 1 hour after the intratracheal administration).

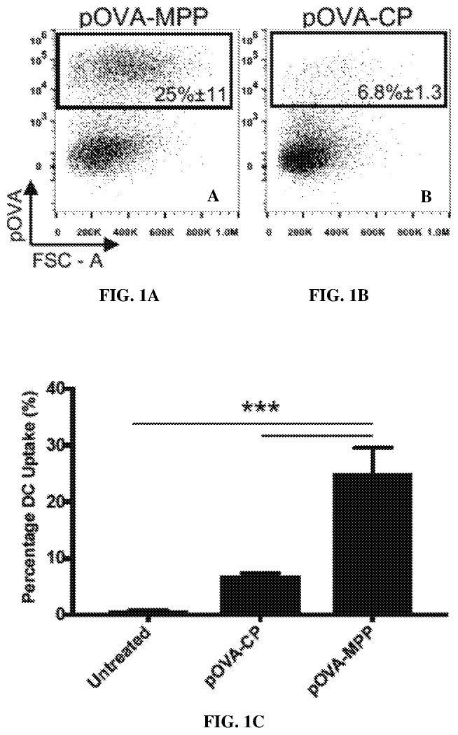

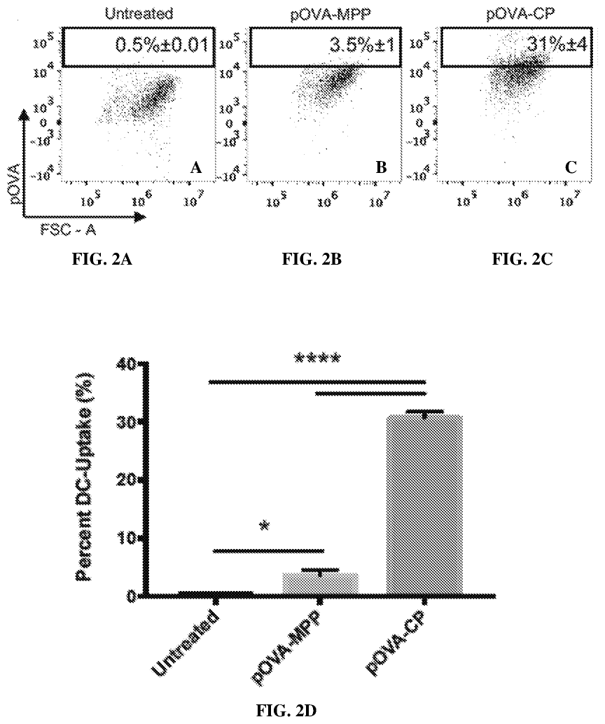

Efficient mucus penetration is critical to particle access to, and subsequent uptake by, pulmonary DC following localized administration into the lung airways. Specifically, female inbred C57BL/6 mice intratracheally received fluorescently-labeled pOVA-MPP or pOVA-CP, and uptake of either formulation by pulmonary DC (CD11⁺CD170⁻) was quantified 16 hours after the administration. Flow cytometric analysis revealed that DC uptake of pOVA-MPP (25%±11%) was significantly greater than that of pOVA-CP (6.8%±1.3%) (FIGS. 1A-1C). In vitro DC uptake of two formulations were compared to test whether the enhanced DC uptake observed with pOVA-MPP in vivo was attributed to its superior DC-targeting and/or endocytic capacity. However, the trend was reversed in vitro where 3.5%±2% and 31%±2% of DC were found positive for pOVA-MPP and pOVA-CP uptake, respectively (FIGS. 2A-2D).

DNA-loaded mucus-penetrating particles (i.e. MPP) was demonstrated to readily reach and internalize into pulmonary DC following intratracheal administration to a markedly greater extent compared to otherwise identical mucus-impermeable conventional particles (i.e. CP). The data shows that the uniquely high mobility in mucus facilitates cross sectional penetration and lateral spread of MPP through the airway mucus gel layer, thereby enhancing the probability of particle encounter and uptake by pulmonary DC. While inhaled CP were found clumped up sparsely and superficially at the very lumen of the gel layer, identically administered MPP exhibited uniform airway distribution near to the epithelial surface. The enhanced MPP uptake by pulmonary DC is also likely due to their ability to circumvent the MCC by rapid and timely mucus penetration (Duncan, et al., Mol. Ther., 24:2043-2053 (2016); Suk, et al., Adv. Drug Deliv. Rev., 99:28-51 (2016)). The intrinsic ability to mediate DC uptake (i.e. in vitro DC uptake) alone appeared insufficient in promoting particle uptake by pulmonary DC in vivo, as evidenced by the negligible DC uptake observed with inhaled CP despite its superior in vitro DC uptake capacity compared to MPP.

TABLE 1
Physicochemical properties of various OVA-loaded MPPs.

| | Hydrodynamic | | |
|---|---|---|---|
| | Diameter ± | | ζ-potential ± |
| Particle Type | SEM^{†}, nm | PDI^{‡} | SEM, mV |
| pOVA-MPP | 55 ± 1 | 0.1 | 1.6 ± 0.3 |
| CpG/pOVA-MPP | 54 ± 1 | 0.1 | 1.7 ± 0.2 |
| p(I:C)/pOVA-MPP | 57 ± 1 | 0.1 | 2.2 ± 0.1 |
| pOVA-CP | 120 ± 4 | 0.1 | 32 ± 2 |

^{†}Hydrodynamic diameter and PDI were measured by dynamic light scattering in water (Ph 7.0). Data represent the mean ± SEM (n ≥ 3).
^{‡}ζ-potential was measured by laser Doppler anemometry in 10 mM NaCl (pH 7.0). Data represent the mean ± SEM (n ≥ 3).

### Example 2. Adjuvant-Loaded pOVA-MPP Efficiently Penetrate Human Airway Mucus Ex Vivo and Activate DC In Vitro

Materials and Methods

DNA-Loaded Nanoparticle Formulation & Characterization

The polymer solution was prepared with PBAE only or a mixture of PBAE and PBAE-PEG (at a wt/wt ratio of 2:3 based on PBAE mass) for CP or MPP, respectively. To engineer DNA-loaded nanoparticles, five volumes of nucleic acids, including labeled or unlabeled plasmid DNA with either CpG or poly(I:C), at 0.1 mg/mL, were added dropwise to one volume of a polymer solution at a PBAE-to-nucleic acid wt/wt ratio of 60:1 while vortexing. DNA-loaded nanoparticles were then washed with five volumes of ultrapure distilled water at 950×g for 8 minutes each time and concentrated to 0.5 mg/mL using Amicon Ultra Centrifugal Filters (100,000 molecular-weight cutoff; Millipore).

To evaluate the ability of MPP to protect nucleic acid payloads, MPP containing 1 μg of pOVA and 0.25 μg of either CpG or poly I:C were treated with 2.5 unit of DNase I (Thermo Fisher Scientific) at 37° C. for 15 minutes. Same amounts of carrier-free pOVA with either CpG or poly I:C were used as controls. Samples were then treated with 365 μg EDTA (Sigma) and further incubated at 65° C. for 10 minutes. To induce de-compaction of the MPP, samples were incubated with heparin (Sigma Aldrich) at a 3:1 (w/w) ratio of heparin to DNA at room temperature for 10 minutes. Samples and 1 kb plus DNA ladder (Thermo Fisher Scientific) were then loaded into a 0.9% agarose gels containing SYBR Safe (Thermo Fisher Scientific), and electrophoresis was conducted sequentially at 50 and 100 V for 10 and 35 minutes, respectively. Finally, Gels were imaged using a ChemiDoc imaging system (Bio-RAD).

In Vitro Dendritic Cell Uptake and Activation Studies

JAWSII cells were plated on a 12-well plate at 200,000 cells/well for both studies. To examine in vitro activation of DC, cells were treated with MPP carrying pOVA without or with CpG or poly I:C at a plasmid-to-adjuvant wt/wt ratio of 5:1. Cells in controls groups were treated with PBS or carrier-free nucleic acids at a same dose. Cells were incubated at 37° C. for 6 hours prior to flow cytometric analysis.

Multiple Particle Tracking

Airway mucus samples were collected from patients visiting the Adult Cystic Fibrosis Center at Johns Hopkins University via spontaneous expectoration under a written informed consent in accordance with the Johns Hopkins Institutional Review Board. The motions of DNA-loaded nanoparticles carrying Cy5-labeled plasmids in the freshly collected mucus samples were captured by high-resolution fluorescent video microscopy and quantified by MPT analysis using a software custom-written in MATLAB (MathWorks), as previously reported (Schuster, et al., Adv. Drug Deliv. Rev., 91:70-91 (2015)).

Results

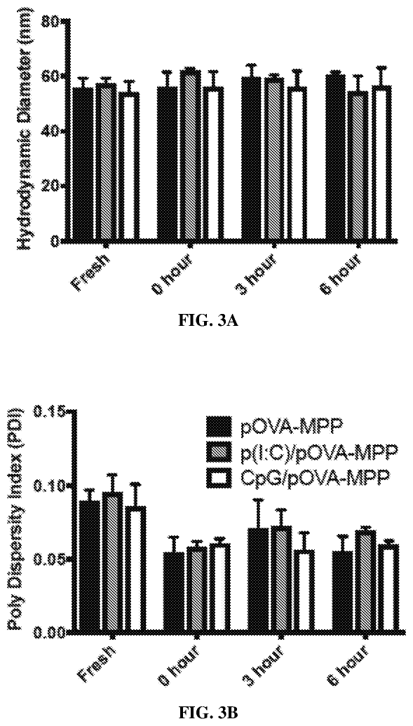

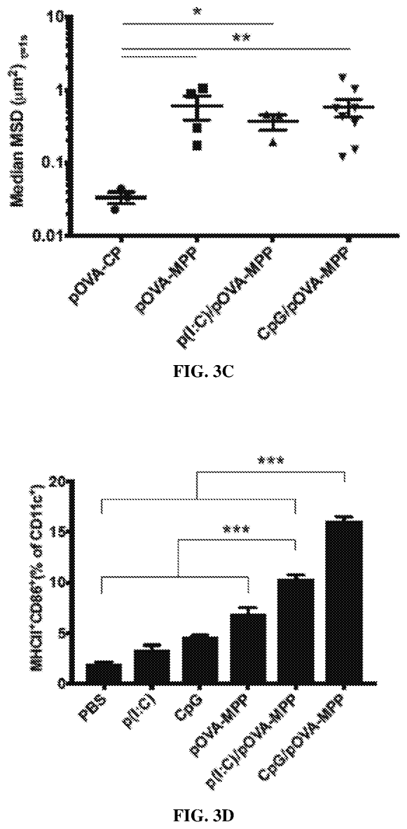

To capitalize the critical roles of adjuvants on vaccination (Bachmann, et al., Nat. Rev. Immunol., 10:787-796 (2010)), pOVA-MPP was formulated with inclusion of short nucleic acid-based adjuvants targeting intracellular toll-like-receptors (TLR); TLR molecules abundant in endocytic compartments of DC promote cross-presentation (Vollmer, et al., Adv. Drug Deliv. Rev., 61:195-204 (2009)). MPP formulations were engineered with a mixture of pOVA and either of p(I:C) (TLR3 agonist) and CpG (TLR9 agonist) and the inclusion of adjuvants did not compromise the mucus-penetrating physicochemical properties of pOVA. Specifically, particle sizes (i.e., hydrodynamic diameters), surface charges and colloidal stability (i.e., changes in hydrodynamic diameters and polydispersity index values in phosphate buffered saline (PBS) over time) of pOVA-MPP co-loaded with p(I:C) (p(I:C)/pOVA-MPP) or CpG (CpG/pOVA-MPP) were virtually identical to those of pOVA-MPP formulated without adjuvants (FIGS. 3A and 3B, and Table 1).

The ability of the MPP formulation to protect nucleic acid payloads against extracellular nucleases was evaluated. Gel electrophoretic analysis revealed that all the payloads, including pOVA, p(I:C) and CpG, remained intact in the formulation following nuclease challenge, unlike carrier-free nucleic acids. Of note, CpG resisted the DNase mediated degradation, regardless of the packaging, due to its intrinsically nuclease-resistant, phosphorothioate backbone (Meng, et al., Bmc Biotechnol., 11 (2011)).

Using multiple particle tracking (MPT) analysis (Schuster, et al., Adv. Drug Deliv. Rev., 91:70-91 (2015)), the impact of the inclusion of adjuvants on the mucus-penetrating property of pOVA-MPP was investigated in airway mucus freshly expectorated from patients visiting the Johns Hopkins Adult Cystic Fibrosis Center. The pathological airway mucus is highly viscoelastic due to mucus build-up and/or chronic infection/inflammation, which is a hallmark of numerous obstructive lung diseases and reinforces the airway mucus as a delivery barrier (Duncan, et al., Mol. Ther., 24:2043-2053 (2016); Kim, et al., J. Control. Release, 240:465-488 (2016)). The MPT measures various transport parameters, such as mean square displacement (MSD); MSD is a measure of the distances traveled by individual particles at a given time interval (i.e., timescale) and thus is directly proportional to particle diffusion rates (Schuster, et al., Adv. Drug Deliv. Rev., 91:70-91 (2015); Lai, et al., Methods Mol. Biol., 434:81-97 (2008); Suh, et al., Adv. Drug Deliv. Rev., 57:63-78 (2005)). All MPP formulations, including pOVA-MPP, p(I:C)/pOVA-MPP, and CpG/pOVA-MPP, exhibited comparably high MSD values (FIG. 3C). In contrast, pOVA-CP were unable to efficiently diffuse in the human airway mucus, displaying significantly lower MSD values compared to all MPP formulations (FIG. 3C).

The abilities of different formulations to induce DC maturation in vitro were investigated. Both p(I:C)/pOVA-MPP and CpG/pOVA-MPP significantly increased the percentage of DC positive for maturation markers (CD86⁺MHC-II⁺); (Herath, et al., Plos One, 9 (2014)) compared to untreated control, carrier-free adjuvants and the adjuvant-free counterpart (i.e., pOVA-MPP) (FIG. 3D). Out of two different adjuvant-loaded MPP formulations, CpG/pOVA-MPP showed the greatest level of matured DC (FIG. 3D), and thus further investigation was conducted with CpG/pOVA-MPP.

### Example 3. Intratracheally Administered CpG/pOVA-MPP Traffic to the Local Lymph Node Via DC and Enhance Effector T-Cell Responses

Materials and Methods

Single Cell Suspension Preparation & Flow Cytometry

Pulmonary immune cells were collected by finely chopping harvested lung tissues, followed by digestion in a media containing 5 mg collagenase D (Worthington) and 1.25 mg of DNase I (Worthington) at 225 rpm in a shaker at 37° C. for 40 minutes. In parallel, immune cells from spleen, Peyer's patches and different LN, including inguinal, mediastinal and mesenteric LN, were isolated by mechanically disrupted respective tissues. Cells were then passed through 70 and 40 μm cell strainers sequentially. Red blood cells from lung and spleen were removed using ammonium-chloride potassium lysing buffer (Thermo Fisher Scientific) according to the manufacturer's instruction. Tissue incubation and washing were done with RPMI 1640 medium (Thermo Fisher Scientific) supplemented with 10% heat-inactivated fetal bovine serum (HI-FBS; Thermo Fisher Scientific).

For flow cytometric experiments, cells were first stained with the LIVE/DEAD fixable dead cell stains kit (Thermo Fisher Scientific) according to the manufacturer's protocol. Cells were then incubated with purified anti-mouse CD16/CD32 antibody (Biolegend) on ice for 10 minutes to block Fc receptors. For the surface staining, cells were incubated with the antibody combination in eBioscience flow cytometry staining buffer (Thermo Fisher Scientific) at 4° C. for 30 minutes. To stain intracellular markers, intracellular fixation & permeabilization buffer set (Thermo Fisher Scientific) was used according to manufacturer's instruction. Antibodies against mouse CD45, CD8, CD3, CD4, CD11c, CD86, IFN-γ, CD11b, CD170, MHC I, MHC II and integrin α₄β₇ were purchased from Thermo Fisher Scientific. The R-phycoerythrin labeled SIINFEKL (SEQ ID NO:1)-MHC I pentamer was purchased from ProImmmune. All the flow cytometric experiments were done using SONY SH800S Cell Sorter and analyzed with the FlowJo Software (FlowJo).

Animal Studies

To evaluate the immune response and memory establishment, mice were immunized and boosted at day 0 and 14, respectively. Mice were sacrificed at day 21 and 70 for assessing immune response and memory establishment, respectively, and relevant tissues were harvested and processed for flow cytometric analysis. For the adoptive transfer study, spleen was harvested from OT-I mice (6-8 weeks; Jackson laboratory) for CD8⁺ T-cell isolation and subsequently enrichment using a negative selection EasySep mouse t-cell isolation kit (STEMCELL Technologies). C57BL/6 mice were then treated with 1×10⁶ splenic CD8⁺ T cells from OT-I mice via a tail vein injection one day prior to the immunization and sacrificed them three days after the immunization for subsequent flow cytometric analysis.

Ex Vivo Re-Stimulation Study

The immunological responsiveness of T-cells from different organs were evaluated by quantifying the intracellular IFN-γ production in the CD8+ and CD4⁺ T-cells isolated from the immunized mice using a previously reported method (Nembrini, et al., P. Natl. Acad. Sci. USA, 108:E989-E997 (2011)). Briefly, single cell suspensions from the lung, spleen and LN harvested 7 days after the boost immunization were prepared, using the procedure described above. Cells were cultured in Iscove modified Dulbecco medium media (Thermo Fisher Scientific) supplemented with 10% HI-FBS, 2 mM L-glutamine and 1% penicillin/streptomycin at 37° C. Cells from spleen and LN were plated on a 6-well plate at a 7×10⁶ cells/well and cultured in presence of monensin (Sigma) and 1 μg/ml of SIINFEKL (SEQ ID NO:1) (InvivoGen) or 100 μg/mL OVA (InvivoGen) for 6 hours to re-stimulate OVA-specific CD8⁺ or CD4⁺ T-cells, respectively. In parallel, cells from the lung were plated in the same manner and re-stimulated with 1 μg/mL ionomycin and 50 ng/ml PMA (InvivoGen) for 4 hours. Cells from all three organs were additionally treated with 5 μg/ml brefeldin A (Thermo Fisher Scientific) for 2 and 3 hours to inhibit secretion of newly produced IFN-γ from CD8⁺ and CD4⁺ T-cells, respectively, prior to the cell collection. Cells were then stained for flow cytometric analysis.

Results

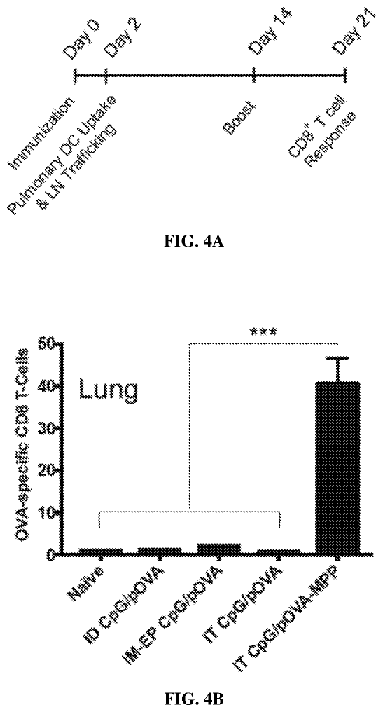

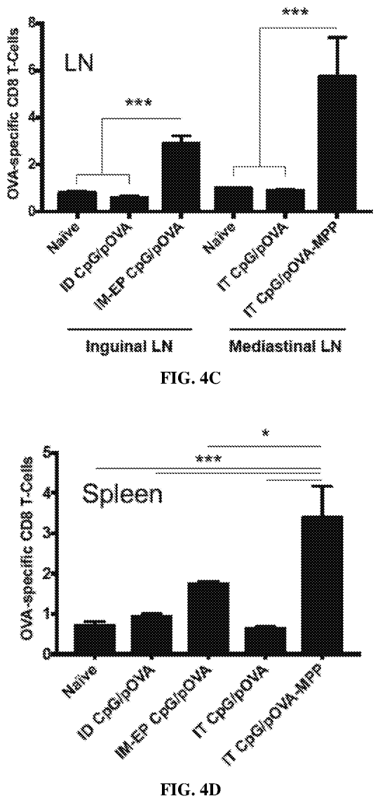

Efficient delivery of DNA vaccines to DC and subsequent migration to lymph node (LN) is critical steps in inducing robust cytotoxic T-cell (CTL) immune response (Moon, et al., Adv. Mater., 24:3724-3746 (2012)). As shown in Examples 1 and 2, pOVA-MPP were efficiently taken up by pulmonary DC in vivo (FIGS. 1A-1C) and adjuvant loaded pOVA-MPP were capable of enhancing DC maturation in vitro (FIG. 3D). Thus, the trafficking of CpG/pOVA-MPP to mediastinal LN was determined using immunohistochemical analysis. Two days after a single intratracheal administration (FIG. 4A), fluorescently-labeled CpG/pOVA-MPP were found co-localized with pulmonary DCs (CD11c⁺CD170⁻) in the lung airway interstitium and subsequently trafficked to mediastinal LN.

Induction of OVA-specific CTL response in vivo following a cycle of immunization and boost with the identical formulation was evaluated (FIG. 4A). The level of OVA-specific CTL response was quantitatively determined by flow cytometric analysis of effector T166 cells (CD3_{E}+CD8⁺) positive for SIINFEKL (SEQ ID NO:1)-MHC I pentamer where SIINFEKL (SEQ ID NO:1) is an antigenic epitope of OVA protein (Nembrini, et al., P. Natl. Acad. Sci. USA, 108:E989-E997 (2011)). Intratracheally (IT) administered CpG/pOVA-MPP showed significantly greater OVA-specific CTL responses in the lung, mediastinal LN and spleen compared to identically administered carrier-free CpG/pOVA that exhibited the responses comparable to those of the naive untreated control (FIGS. 4B-4D). IT CpG/pOVA171 MPP with carrier-free CpG/pOVA administered via other conventional delivery routes were compared, including intradermal (ID) injection or intramuscular injection followed by electroporation (IM-EP). IT CpG/pOVA-MPP exhibited significantly greater OVA-specific CTL responses compared to both ID CpG/pOVA and IM-EP CpG/pOVA in all three different tissues, including the lung, respective LN (i.e., mediastinal and inguinal LN for IT and ID/IM-EP, respectively) and spleen (FIGS. 4B-4D). In particular, IT CpG/pOVA-MPP resulted in ˜40% of OVA-specific CTL in the lung, unlike all other control groups that exhibited negligible levels of pulmonary CTL responses.

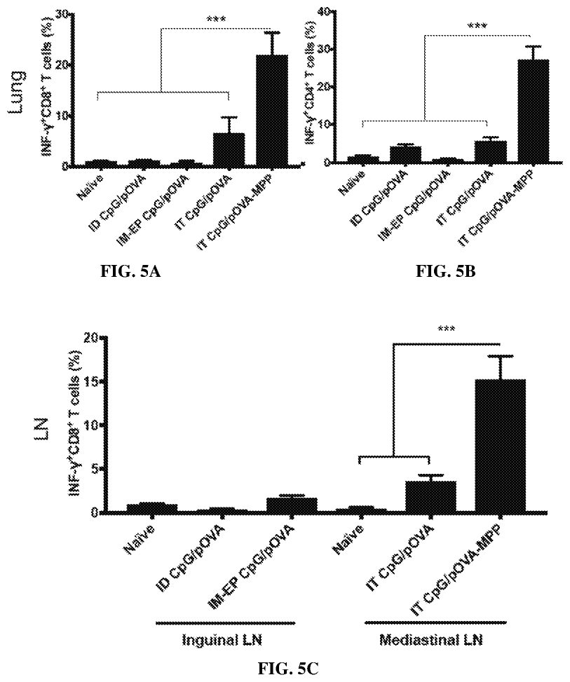

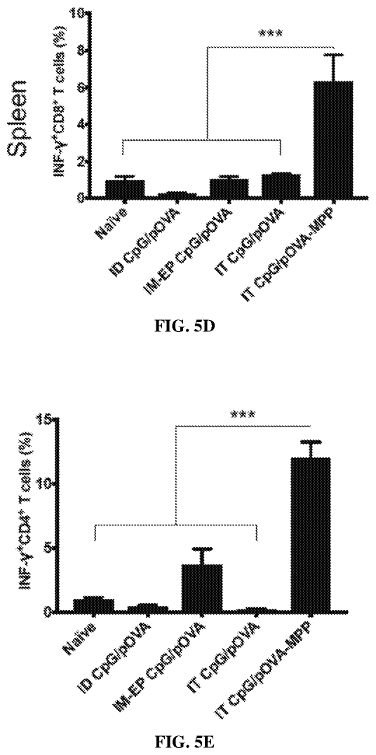

Immune responses were analyzed by evaluating the responsiveness of OVA-specific helper (CD4⁺) and effector (CD8⁺) T-cells to ex vivo re-stimulation. Specifically, CD4⁺ and CD8⁺ T-cells in the lung (FIGS. 5A and 5B), respective LN (FIG. 5C) and spleen (FIGS. 5D and 5E) were harvested 7 days after the boost, re-stimulated ex vivo with phorbol myristate acetate (PMA)/ionomycin or SIINFEKL (SEQ ID NO:1) peptide (Nembrini, et al., P. Natl. Acad. Sci. USA, 108:E989-E997 (2011)), and T-cells producing interferon-g (IFN-g) were quantified using flow cytometry. Similar to the observation with the in vivo CTL response study (FIGS. 4B-4D), IT CpG/pOVA-MPP group exhibited the greatest frequency of activated CD8⁺ T-cells (IFN-g⁺CD8⁺) uniformly in all three different tissues (FIGS. 5A-5E). IT CpG/pOVA-MPP yielded significantly greater frequencies of pulmonary (FIGS. 5A and 5B) and splenic (FIGS. 5D and 5E) CD4⁺ T-cell activation (i.e., percentage of IFN-g⁺CD4⁺ T-cells) upon ex vivo re-stimulation, compared to all other test conditions, including IT CpG/pOVA, ID CpG/pOVA and IM-EP CpG/pOVA groups.

Inhaled MPP carrying DNA vaccine components were efficiently taken up by pulmonary DC resided in the lung interstitium, trafficked to the local lymph node and subsequently elicited strong pulmonary immunity. MPP carrying both antigen-expressing plasmids and nucleic acid-based adjuvants (i.e., CpG/pOVA-MPP) was designed and the formulation mediated significantly stronger and more durable adaptive immunity in the lung.

Inhaled MPP-mediated vaccination provided greater systemic immunity than dose-matched antigen-expressing plasmids and adjuvants co-administered via routes commonly applied for systemic vaccination. The findings underline an important role of improving the access to DC, in addition to more widely explored strategies to augment DC uptake, on achieving robust mucosal and potentially systemic immunity.

Previous studies, such as Bivas-Benita, et al. and Li, et al., reported greater antigen-specific immunity by inhaled over standard systemic vaccination approaches (Bivas-Benita, et al., J. Virol., 84:5764-5774 (2010); Li, et al., Sci. Transl. Med, 5:204ra130 (2013)). However, previous studies only showed that inhaled nanoparticle-based DNA vaccination is capable of inducing a robust systemic immunity but to a level comparable to that achieved by intramuscular immunization (Bivas-Benita, et al., J. Virol., 84:5764-5774 (2010)). The enhanced systemic immunity by inhaled MPP demonstrated here was not expected a priori. The high in vivo DC uptake of MPP (i.e. ˜25% of overall pulmonary DC) observed here accounts for the greater systemic immunity. Number of DC accessible to DNA vaccine components administered via conventional intradermal and intramuscular routes is limited, leading to suboptimal systemic immunity.

### Example 4. Intratracheal Immunization with CpG/pOVA-MPP Mediates OVA-Specific Effector CD8⁺ T-Cell Dissemination to Distal Mucosal Tissues

Materials and Method

To assess the capacity of IT CpG/pOVA-MPP to induce OVA-specific CTL responses in distal mucosal tissues, CD8⁺ T-cells harvested from gastrointestinal (GI) and vaginal tract after intratracheal immunization were analyzed. Mice were immunized as scheduled in FIG. 4A and the OVA-specific effector CD8⁺ T-cells positive for SIINFEKL (SEQ ID NO:1)-MHC I pentamer in mesenteric LN and Peyer's patches in GI as well as the whole vagina were quantified using flow cytometry 7 days after the boost.

Vaginal immune cells were collected by finely chopping harvested vaginal tissues, followed by digestion in a media containing 4 mg collagenase (Sigma) and 1.25 mg of DNase I (Worthington) at 225 rpm in a shaker at 37° C. for 2 hours. In parallel, immune cells from Peyer's patches and mesenteric LN, were isolated by mechanically disrupted respective tissues. Cells were then passed through 70 and 40 μm cell strainers sequentially. Red blood cells from lung and spleen were removed using ammonium-chloride potassium lysing buffer (Thermo Fisher Scientific) according to the manufacturer's instruction. Tissue incubation and washing were done with RPMI 1640 medium (Thermo Fisher Scientific) supplemented with 10% heat-inactivated fetal bovine serum (HI-FBS; Thermo Fisher Scientific).

For flow cytometric experiments, cells were first stained with the LIVE/DEAD fixable dead cell stains kit (Thermo Fisher Scientific) according to the manufacturer's protocol. Cells were then incubated with purified anti-mouse CD16/CD32 antibody (Biolegend) on ice for 10 minutes to block Fc receptors. For the surface staining, cells were incubated with the antibody combination in eBioscience flow cytometry staining buffer (Thermo Fisher Scientific) at 4° C. for 30 minutes. To stain intracellular markers, intracellular fixation & permeabilization buffer set (Thermo Fisher Scientific) was used according to manufacturer's instruction. Antibodies against mouse CD45, CD8, CD3, CD4, CD11c, CD86, IFN-γ, CD11b, CD170, MHC I, MHC II and integrin α₄β₇ were purchased from Thermo Fisher Scientific. The R-phycoerythrin labeled SIINFEKL (SEQ ID NO:1)-MHC I pentamer was purchased from ProImmmune. All the flow cytometric experiments were done using SONY SH800S Cell Sorter and analyzed with the FlowJo Software (FlowJo).

Results

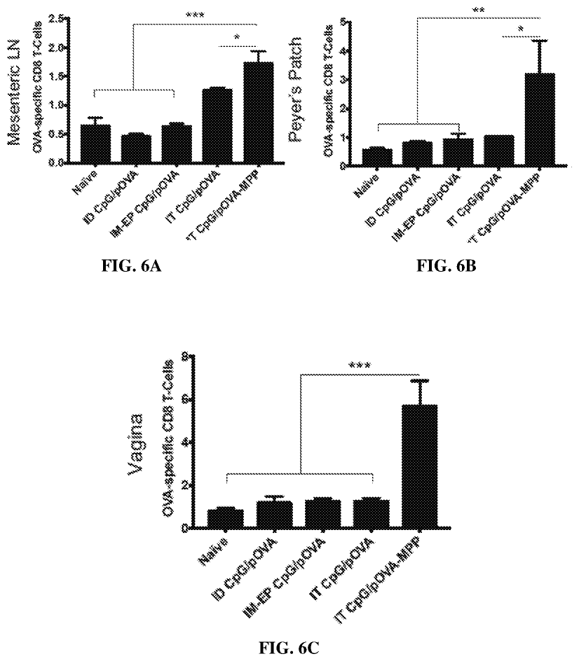

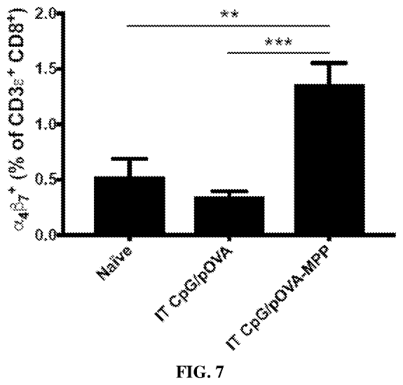

IT CpG/pOVA-MPP with other carrier-free and/or conventional route controls were compared, including IT CpG/pOVA, ID CpG/pOVA and IM-EP CpG/pOVA groups. IT CpG/pOVA-MPP group exhibited significantly greater frequencies of OVA-specific CD8⁺ T-cells both in GI and vaginal tracts compared to all the control groups (FIGS. 6A-6C). IT CpG/pOVA-MPP significantly increased the percentage of CD8⁺ T-cells (CD3_{E}+CD8⁺) harvested from the mediastinal LN that express a gut-homing integrin α₄β₇ compared to the carrier-free IT CpG/pOVA group 7 days after the boost immunization (FIG. 7).

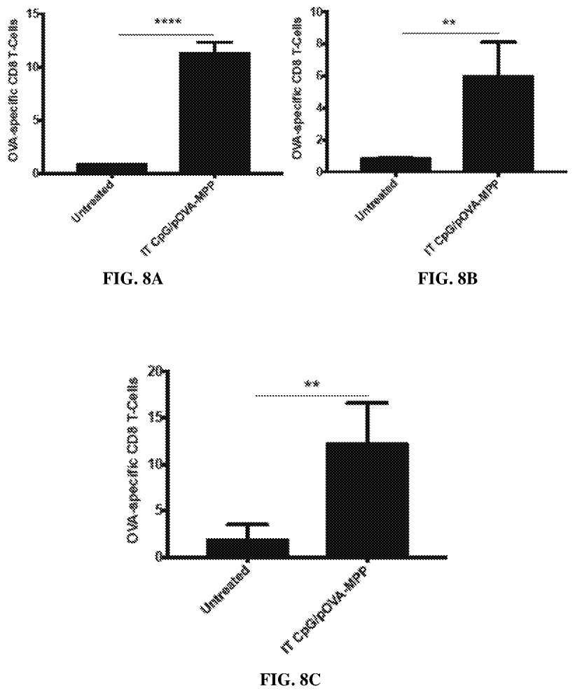

Dissemination of OVA-specific CD8⁺ T-cells to distal mucosal tissues by IT CpG/pOVA-MPP was confirmed using an adoptive T-cell transfer study. Specifically, C57BL/6 mice received an intravenous injection of CD8⁺ T-cells harvested from OT-I mice and subsequently immunized with IT CpG/pOVA-MPP or left untreated on the following day. Three days after the immunization, significantly greater frequencies of OVA-specific CD8⁺ T-cell response were detected via flow cytometry in both GI and vaginal tracts compared to the untreated control (FIGS. 8A-8C).

MPP carrying both antigen-expressing plasmids and nucleic acid-based adjuvants (i.e., CpG/pOVA-MPP) mediated significantly stronger and more durable adaptive immunity in remote mucosal surfaces compared to conventional systemic DNA vaccinations. Pulmonary DNA vaccination by MPP led to robust trans-mucosal antigen-specific CTL responses both in GI and vaginal tracts. This is attributed to the ability of this vaccination approach to promote crosstalk between the lung and distal mucosal surfaces, as evidenced by the elevation of CD8⁺ T-cells expressing a gut-homing integrin in the local LN of the lung (i.e. mediastinal LN) (Ruane, et al., J. Exp. Med., 210:1871-1888 (2013)). Of note, carrier-free DNA vaccination given intratracheally (i.e. IT CpG/OVA) was unable to induce antigen-specific CTL responses in remote mucosal surfaces, highlighting the key contribution of MPP formulation on establishing trans-mucosal immunity.

### Example 5. Intratracheal Immunization with CpG/pOVA-MPP Establishes OVA-Specific Long-Term CD8⁺ T-Cell Responses and Effector Memory—Biased Immunity in the Lung

Materials and Methods

The long-term OVA-specific CTL response mediated by IT CpG/pOVA-MPP in comparison to the carrier-free IT pOVA-MPP control 70 days after the immunization was assessed.

Results

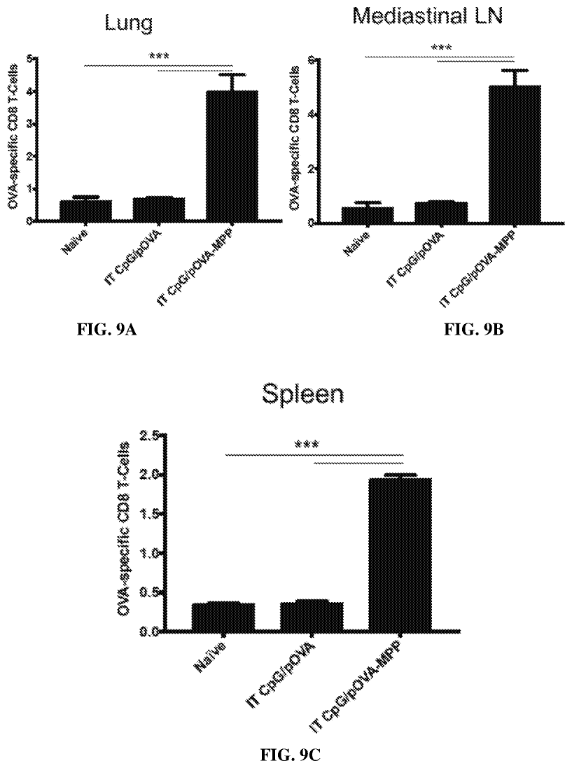

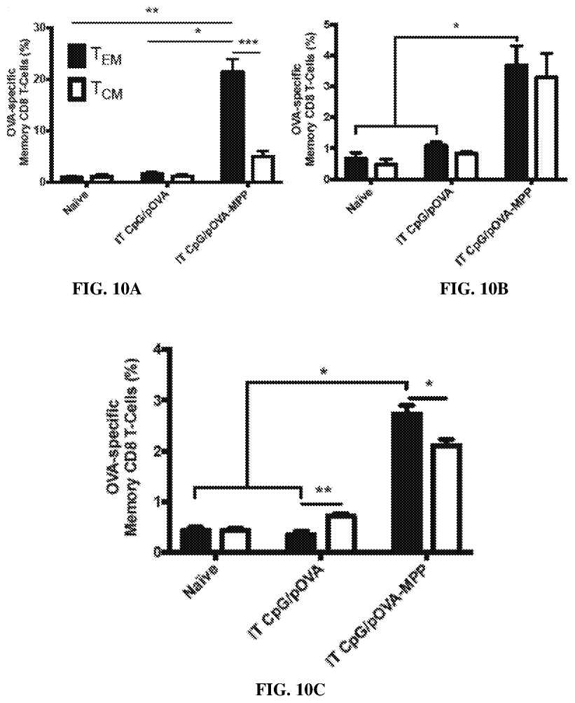

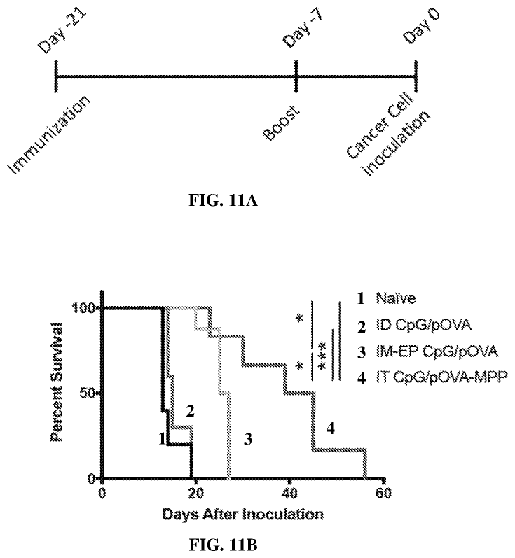

It is critical to establish antigen-specific memory to achieve a long-term protective or therapeutic immunity. Similar to the observation at 21-day post-immunization (i.e. 7 days after the boost) (FIGS. 4B-4D), the MPP formulation significantly increased the percentage of OVA-specific CD8⁺ T222 cells in the lung, mediastinal LN and spleen (FIGS. 9A-9C). To confirm the establishment of OVA223 specific CD8⁺ T-cell memory, the cells for the expression of memory associated surface markers were analyzed, including CD44 and CD62L (van Faassen, et al., J. Immunol., 174:5341-5350 (2005)). Of note, central (T_{CM}) and effector (T_{EM}) memory T-cells are distinguished by relative expression of CD62L where T_{CM} and T_{EM} exhibit CD44^{hi}CD62L^{hi} and CD44^{hi}CD62L^{lo}, respectively (van Faassen, et al., J. Immunol., 174:5341-5350 (2005)). At 70 days post-immunization that IT CpG/pOVA-MPP markedly increased both memory phenotypes compared to carrier-free IT CpG/pOVA control in the lung, mediastinal LN and spleen (FIGS. 10A-10C). In addition, IT CpG/pOVA-MPP established OVA-specific T-cell memory strongly biased towards the effector memory phenotype both at the site of administration (i.e., lung) and systemically in the spleen (FIGS. 10A-10C). In particular, the bias was most prominent in the lung with over 4-fold greater frequency of T_{EM} compared to that of T_{CM}.

The pulmonary vaccination approach mediates long-lasting CTL and memory T-cell responses in an antigen-specific manner both locally in the lung and systemically. Importantly, T_{EM}-biased response mediated by inhaled MPP is particularly pronounced in the lung, whereas T_{CM}-biased response is generally observed with conventional vaccination (i.e. electroporation) (Rosati, et al., Vaccine, 26:5223-5229 (2008)). T_{CM} must undergo multiple steps upon encountering pathogens, including activation, expansion, differentiation and trafficking, to initiate significantly-delayed effector responses (Robinson, et al., Nat. Med., 11:S25-32 (2005)). In contrast, T_{EM} in the lung, readily available by the MPP-mediated DNA vaccination, immediately acts on and rapidly removes respiratory pathogens (Harari, et al., J. Virol., 83:2862-2871 (2009)), thereby efficiently preventing their replication at the early stage of infection (Shafiani, et al., J. Exp. Med., 207:1409-1420 (2010); Hansen, et al., Nat. Med., 15:293-299 (2009)). The result is due to the ability of MPP to enhance the DNA vaccine uptake by pulmonary DC, which leads to more profound and durable antigen presentation to T-cells (Banchereau, et al., Nature Reviews Immunology, 5:296-306 (2005)).

Albeit to a lesser extent, inhaled MPP induced T_{EM}-biased response in the spleen as well, potentially providing a means to elicit fast-acting systemic immunity.

### Example 6. Intratracheally Immunization with CpG/pOVA-MPP Enhances Anti-Cancer Effect in an Orthotopic Mouse Model of Aggressive Lung Cancer

Materials and Methods

For the anti-cancer efficacy study, an orthotopic mouse model of lung cancer was established by intratracheal inoculation of C57BL/6 mice with 1×10⁶ OVA cells expressing SIINFEKL (SEQ ID NO:1) 7 days prior to boost immunization and monitored survival of mice over time.

C57BL/6 mice were first immunized following the schedule in FIG. 11A, inoculated mice intratracheally with highly aggressive and poorly immunogenic (Bellelli, et al., Tumori, 68:373-380 (1982); Lechner, et al., J. Immunother., 36:477-489 (2013)) Lewis lung carcinoma (LLC) cells expressing SIINFEKL (SEQ ID NO:1), and monitored their survival over time (FIG. 11A).

Results

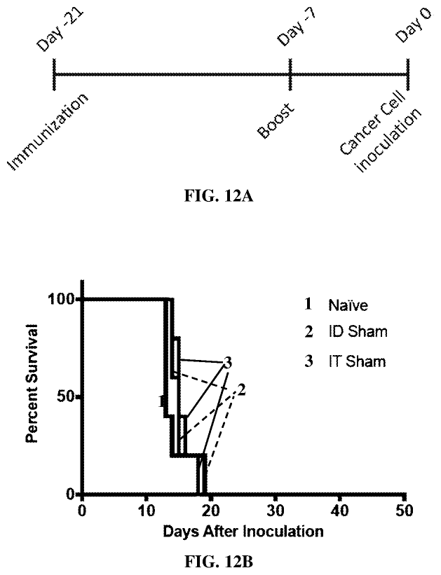

The pulmonary vaccination with CpG/pOVA-MPP provided an enhanced anti-cancer effect in an orthotopic syngeneic mouse model of lung cancer in comparison to conventional route (i.e. ID CpG/pOVA and IM-EP CpG/pOVA) controls. While the survival of mice received ID CpG/pOVA were comparable to that of untreated naive mice, IM-EP CpG/pOVA was able to significantly enhance the survival compared to these two groups (FIG. 11B). However, IT CpG/pOVA-MPP group exhibited by far greatest survival compared to all other groups; the median survival days of mice in the IT CpG/pOVA-MPP, IM-EP CpG/pOVA and ID CpG/pOVA groups and of untreated mice were 42, 26, 15 and 13 days, respectively (FIG. 11B). Sham administration via intradermal and intratracheal routes did not affect the survival of the animal model (FIGS. 12A and 12B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A mucus penetrating nanoparticle comprising
a core comprising a blend of a biodegradable hydrophobic polymer and a hydrophilic or amphiphilic,
wherein ≥50% of the hydrophilic polymer is conjugated to the biodegradable hydrophobic polymer, and
the nanoparticle is coated with the hydrophilic polymer,
wherein the core encapsulates nucleic acid encoding polypeptide antigen and/or the polypeptide antigen.

2. The nanoparticle of claim 1, wherein the core comprises a nucleic acid encoding the polypeptide antigen.

3. The nanoparticle of claim 2, wherein the antigen is a T cell antigen.

4. The nanoparticle of claim 2, wherein the nucleic acid is RNA or DNA.

5. The nanoparticle of claim 4, wherein the nucleic acid is DNA.

6. The nanoparticle of claim 5, wherein the DNA is a DNA vector encoding a heterologous expression control sequence operably linked to a sequence encoding the polypeptide antigen.

7. The nanoparticle of claim 6, wherein the vector is a plasmid or viral vector.

8. The nanoparticle of claim 7, wherein the vector is a plasmid.

9. The nanoparticle of claim 1, wherein the nanoparticle further comprises an adjuvant.

10. The nanoparticle of claim 9, wherein the adjuvant is selected from the group consisting of ligands for pattern recognition receptors (PPRs), adaptor proteins, inflammation singling proteins, transcription factors, cytokines, chemokines, immune costimulatory molecules, toll-like receptor agonists or inhibitors of immune suppressive pathways, and immune regulators, or a nucleic acid encoding any of foregoing.

11. The nanoparticle of claim 9, wherein the adjuvant is a ligand for a PPR.

12. The nanoparticle of claim 11, wherein the PPR is a Toll-like family member.

13. The nanoparticle of claim 12, wherein the adjuvant acts through TLR3, TLR4, TLR5, TLR6, TLR7, TLR8, TLR9, or a combination thereof.

14. The nanoparticle of claim 9, wherein the adjuvant is an oligonucleotide comprising one or more unmethylated cytosine-guanine (CpG) dinucleotide motifs.

15. The nanoparticle of claim 14, wherein the adjuvant is Poly(I:C) or a derivative thereof.

16. The nanoparticle of claim 1, wherein the mass ratio of the free biodegradable polymer to the conjugated biodegradable hydrophobic polymer is between 0.5 and 1, based on the mass of the biodegradable polymer.

17. The nanoparticle of claim 2, wherein the mass ratio of the blended polymer to nucleic acid is up to 100.

18. The nanoparticle of claim 1 comprising nucleic acid and adjuvant, wherein the mass ratio of nucleic acid to adjuvant is up to 10.

19. The nanoparticle of claim 1, wherein the hydrodynamic diameter of the nanoparticle is less than 100 nm.

20. The nanoparticle of claim 1, wherein the surface charge of the nanoparticle is near neutral.

21. The nanoparticle of claim 1, wherein the biodegradable polymer is poly(β-amino ester) with a molecular weight between 4 kDa and 7 kDa.

22. The nanoparticle of claim 1, wherein the hydrophilic polymer is a polyalkylene oxide or copolymer thereof.

23. The nanoparticle of claim 1, wherein the hydrophilic polymer is polyethylene glycol with a molecular weight between 1 kDa and 10 kDa.

24. The nanoparticle of claim 1, further comprising an immunomodulatory agent selected from the group consisting of synthetic receptor ligands, proteins, cytokines, interleukins, tumor necrosis factor, and combinations thereof.

25. An immunogenic composition comprising the nanoparticles according to claim 1, wherein the nanoparticles are in an amount effective to induce an immune response in a subject in need thereof.

26. The composition of claim 25, wherein the composition increases antigen uptake in pulmonary dendritic cells (DC) (CD11⁺CD170⁻); increases DC maturation; increases DC number or frequency, particularly pulmonary DCs (CD11c⁺CD170⁻) in the lung airway interstitium; increases DC migration to the lymph nodes; increases antigen-specific CTL response, particularly in the lung, mediastinal LN and/or spleen; increases activated CD8⁺ T-cells (IFN-g⁺CD8⁺) and/or increases frequencies of CD4⁺ T-cell activation, particularly in the lung, mediastinal LN and/or spleen; increases dissemination of antigen-specific CD8⁺ T cells to, and/or CTL responses in, tissues distal to the site of administration; increases antigen specific T-cell memory biased towards the effector memory phenotype both at the site of administration and/or systemically in the spleen, preferably wherein the bias is most prominent in the lung; increases gut homing integrin (alpha4beta7) in the CD8+ T cells in mediastinal lymph node; or a combination thereof.

27. The composition of claim 25, wherein the nanoparticles are formulated for administration to a mucosal layer.

28. The composition of claim 26, wherein the nanoparticles are formulated for pulmonary administration.

29. The composition of claim 26, wherein the nanoparticles are taken up by pulmonary dendritic cells and subsequently traffic to lymph node.

30. The composition of claim 25, wherein the nanoparticles comprise antigen expressing DNAs and nucleic acid-based adjuvants.

31. The composition of claim 25, comprising an adjuvant.

32. The composition of claim 31, wherein the adjuvant is loaded into the same nanoparticles as the antigen or nucleic acid encoding the antigen, into different nanoparticles from the antigen or nucleic acid encoding the antigen, or a combination thereof.

33. The composition of claim 31, wherein the adjuvant is not loaded into nanoparticles.

34. A method of inducing an immune response in a subject comprising
administering to the respiratory tract of a subject in need thereof the immunogenic composition of claim 25.

35. The method of claim 34, wherein the composition is administered to a mucosal layer.

36. The method of claim 34, wherein the nanoparticles comprise DNA vector encoding the antigen.

37. The method of claim 34, further comprising administering the subject an adjuvant to the subject.

38. The method of claim 37, wherein the adjuvant is present in the nanoparticles.

39. The method of claim 34, wherein the composition increases adaptive immunity in the lung and other remote mucosal surfaces selected from the group consisting of gastrointestinal tract, vaginal tract, and a combination thereof.

40. The method of claim 34, wherein the composition increases systemic immunity.

41. The method of claim 34, wherein the subject has cancer or an infection, and wherein the immune response is against the cancer or infection.

42. The method of claim 41, wherein the cancer is a lung cancer, or the infection is a lung infection.
